(19) United States
(12) Patent Application Publication
(10) Pub. No.: US 2022/0283172 A1
(43) Pub. Date: Sep. 8, 2022

(54) REDUCE DISCARD OF KIDNEYS FOR TRANSPLANATION AFTER BRAIN DEATH

(71) Applicant: THE JOHNS HOPKINS UNIVERSITY, Baltimore, MD (US)

(72) Inventor: Chirag Parikh, Baltimore, MD (US)

(21) Appl. No.: 17/633,292

(22) PCT Filed: Aug. 7, 2020
(86) PCT No.: PCT/US2020/045405

§ 371 Date: Feb. 7, 2022

Related U.S. Application Data
(60) Provisional application No. 62/883,763, filed on Aug. 7, 2019.

(51) Int. Cl. G01N 33/68; G01N 33/543

(52) U.S. Cl. CPC G01N 33/68; G01N 33/54388

(57) ABSTRACT

The present invention relates to the field of organ transplantation. More specifically, the present invention provides compositions and methods useful for reducing the discard of kidney for transplantation after brain death. In a specific embodiment, a method for assessing viability of a kidney for transplantation comprises the step of measuring the amount of UMOD and OPN in a urine sample obtained from a deceased donor using a point-of-care lateral flow device, wherein ratio of UMOD:OPN of ≤3 indicates the kidney is viable for transplantation.

### CROSS-REFERENCE TO RELATED APPLICATIONS

This application claims the benefit of U.S. Provisional Application No. 62/883,763, filed Aug. 7, 2019, which is incorporated herein by reference in its entirety.

### GOVERNMENT SUPPORT CLAUSE

This invention was made with government support under grant no. DK093770, awarded by the National Institutes of Health. The government has certain rights in the invention.

### FIELD OF THE INVENTION

The present invention relates to the field of organ transplantation. More specifically, the present invention provides compositions and methods useful for reducing the discard of kidney for transplantation after brain death.

### BACKGROUND OF THE INVENTION

Deceased donors undergo extensive biological changes with simultaneous activation of both injury and recovery processes in response to ischemia.¹ As neurogenic hypotension and systemic up-regulation of pro-inflammatory markers follow brain death (the predominant process by which death occurs in deceased donors in the United States), there is direct renal injury secondary to ischemia and reperfusion as well as upregulation of inflammatory pathways in the kidneys.²⁻⁴ Along with injury and inflammation, there is also initiation of both adaptive and maladaptive processes in the kidney.^{5,6} The present inventors hypothesize that these adaptive and maladaptive mechanisms are initiated in donor kidneys, but have a durable effect on subsequent graft function with resolution of parenchymal injury and favorable long-term sequelae vs. accelerated fibrosis and reduction in graft function, respectively. Interrogating these pathways may aid in predicting recipient graft function, which would help further risk stratify deceased-donor kidneys for appropriate allocation. Markers such as YKL-40 (also known as CHI3L1) have been shown to have a protective effect on recipient graft failure and 6-month graft function.⁶ These findings indicate the need to investigate other related biological pathways that may affect the trajectory of kidney allograft function after transplantation.

### SUMMARY OF THE INVENTION

Deceased donor kidneys undergo extensive injury, activating adaptive and maladaptive pathways. Uromodulin (UMOD) and osteopontin (OPN) are two tubular epithelial proteins whose production is induced in the kidney following ischemia. The association of these proteins with kidney transplant outcomes has yet to be investigated.

In the Deceased Donor Study described herein, the present inventors measured urine UMOD and OPN levels from 1298 deceased donors at organ procurement and determined their association with donor acute kidney injury (AKI). The present inventors followed 2430 kidney recipients for a primary outcome of death-censored graft failure (dcGF) and secondary outcome of all-cause graft failure (GF). The present inventors split the data into training and test datasets to develop and evaluate the ratio of UMOD and OPN and its association with dcGF and GF.

AKI occurred in 322 (25%) donors. During a median (IQR) follow-up of 4 (3, 5) years, 13% experienced dcGF [33 (30-37) per 1000 patient-years] and 26% recipients experienced GF [66 (61-71) per 1000 patient-years)]. Each doubling of urine UMOD concentration was independently associated with 28% lower odds of donor AKI [adjusted odds ratio, aOR (95% CI) 0.72 (0.64-0.81)]. However, each doubling of UMOD was independently associated with increased risk for recipient dcGF with an adjusted hazard ratio (aHR) of 1.1 (1.02-1.2). In contrast, each doubling of OPN was independently associated with increased odds of donor AKI [aOR 1.18 (1.09-1.28)] and decreased risk of dcGF [aHR 0.94 (0.88-1)]. UMOD and OPN associations were similar for all-cause GF. A ratio of UMOD to OPN ≤3 was independently and significantly associated with decreased risk of dcGF [aHR 0.57 (0.41-0.80)], with similar findings in the test dataset.

In this large deceased-donor cohort, the present inventors found that the ratio of UMOD to OPN may help characterize the maladaptive and adaptive processes in deceased-donor kidneys. Accordingly, in one aspect, the present invention provides compositions and methods for measuring UMOD and OPN.

Accordingly, in one aspect, the present invention provides a method comprising the step of measuring the amount of uromodulin (UMOD) and osteopontin (OPN) in a urine sample obtained from a deceased person. In one embodiment, the method further comprises the step of measuring the amount of YKL-40. In particular embodiments, the measuring step is performed using a lateral flow device.

In another aspect, the present invention provides methods for assessing viability of a kidney for transplantation. In a specific embodiment, the method comprises the step of measuring the amount of UMOD and OPN in a urine sample obtained from a deceased donor using a point-of-care lateral flow device, wherein ratio of UMOD:OPN of ≤3 indicates the kidney is viable for transplantation and a ratio of UMOD:OPN of ≥3 indicates the kidney is not viable for transplantation. In a more specific embodiment, the lateral flow device is a dipstick assay. In other embodiments, the method is performed using polymerase chain reaction (PCR). It is understood that the ratio of UMOD to OPN can range from about 2 to about 4, wherein ratios less than or equal to such recited amount or range indicates kidney viability.

In another embodiment, a method for assessing viability of a kidney of a deceased donor for transplantation comprising the steps of (a) contacting a urine sample obtained from a deceased donor with a first antibody and a second antibody on a lateral flow device, wherein the first antibody specifically binds UMOD and the second antibody specifically binds OPN; and (b) detecting the amount of UMOD and OPN, wherein a ratio of UMOD:OPN of ≤3 indicates the kidney is viable for transplantation and ratio of UMOD:OPN of ≥3 indicates the kidney is not viable for transplantation. In a more specific embodiment, the lateral flow device is a dipstick assay.

In a further aspect, the present invention provides kits for assessing kidney viability for transplantation. In one embodiment, the kit comprises (a) a first capture agent that specifically binds UMOD present in a sample obtained from the kidney donor; (b) a second capture agent that specifically binds OPN present in a sample obtained from the kidney donor; and (c) instructions for performing a method of assessing kidney viability for transplantation. In certain embodiments, the kidney donor is deceased. In other embodiments, the sample is a urine sample. In other embodiments, the kit further comprises a solid support on which to perform the assay. In particular embodiments, the first capture agent and the second capture agent are or are capable of being detectably labeled. The kits of the present invention can further comprise (d) a first detection agent that detects the first capture agent bound to UMOD; and (e) a second detection agent that detect the second capture agent bound to OPN.

In another embodiment, the kit can further comprise a third capture agent that specifically binds YKL-40 present in a sample obtained from the kidney donor. In a specific embodiment, the third capture agent is or is capable of being detectably labeled. In a more specific embodiment, the kit further comprises a third detection agent that detects the third capture agent bound to YKL-40.

In yet another aspect, the present invention provides a lateral flow multiplex assay device. In one embodiment, the lateral flow device comprises (a) a sample pad configured to receive a urine sample from a deceased kidney donor; (b) a conjugate pad comprising a first detection conjugate comprising an agent that specifically binds UMOD and a second detection conjugate comprising an agent that specifically binds OPN; and (c) at least one detection zone comprising a test line.

In certain embodiments, the first detection conjugate and the second detection conjugate are antibodies. In other embodiments, the antibodies are biotinylated. In further embodiments, the test line comprises immobilized streptavidin particles.

### BRIEF DESCRIPTION OF THE FIGURES

FIG. 1. Median (IQR) levels of donor UMOD and OPN by donor AKI.

FIG. 2. Associations of UMOD and OPN with donor AKI and death-censored graft failure.

FIG. 3. Kaplan Meier plot of death-censored graft failure by UMOD/OPN ratio in the training dataset. The survival curve shows that deceased donor kidneys with lower UMOD to OPN ratio have better graft survival with a log-rank p-value of 0.0016. The numbers below in red and blue show the population at risk at each event time with red representing donor urine with UOMD to OPN ratio >3 and blue representing UMOD to OPN ratio ≤3. Primary non-function was included as survival time of zero.

FIG. 4A-4B. Dual staining of UMOD and OPN from deceased donor biopsies by AKI status. FIG. 4A: dual stain for uromodulin (UMOD, red) and osteopontin (OPN, teal) shows limited staining mostly in the loop of Henley in control tissues and osteopontin is negative (n=4).

FIG. 4B: Tubular casts and injured tubules including proximal tubules and loop of Henley stain for OPN and UMOD in deceased donor biopsies showing acute tubular injury (n=6). 40×, insets show zoom.

FIG. 5. Inclusion criteria for the Deceased Donor Study.

FIG. 6. Antibody affinity measurement: capture antibody-ligand interaction.

FIG. 7. Antibody affinity measurement: antibody pairing interaction.

FIG. 8. Schematic of a standard lateral flow test strip.

FIG. 9. ELISA assessment of antibody pairings.

FIG. 10. ELISA assessment of additional antibody pairings.

FIG. 11. Schematic of Biotin/PSA capture format.

FIG. 12. Evaluation of biotin/PSA format (wet testing). *test line is visible despite low readings.

FIG. 13. OPN standard curve: capture antibody plotted at 0.5 mg/ml.

FIG. 14. Lateral flow testing in urine samples. *test line is visible despite low readings.

FIG. 15. UMOD urine ELISA.

FIG. 16. CHI3-L1 urine ELISA.

FIG. 17. OPN lateral flow standard curve.

FIG. 18. Urine testing in lateral flow; spike recovery.

FIG. 19. Standard curves of OPN, UMOD and YKL-40 assay. OPN, UMOD and YKL-40 yielded favorable results with lo non-specific binding and acceptable dynamic ranges. Assay ranges: OPN 5-250 ng/m1; UMOD 10-10000 ng/ml; and YKL-40 10-500 ng/ml. IL-9 and TNF-α had low sensitivity and were not developed further.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Uromodulin (UMOD), also known as the Tamm-Horsfall protein, and osteopontin (OPN) have been shown to be synthesized by renal tubular epithelial cells and to play important roles in normal physiology and in response to renal injury.^{7,8} UMOD is produced exclusively by the kidney, primarily in the epithelial cells of the thick ascending limb,^{9,10} and has a molecular weight of 100 kDa.¹¹ It is the major component of hyaline casts and the most abundant protein found in urine.¹² UMOD aggregates can serve as ligands to help activate the innate immune response and induce an inflammatory response with activation of tumor necrosis factor-alpha (TNF-alpha) and granulocytes.¹³⁻¹⁵ Furthermore, UMOD has been associated with the incident development of CKD.¹⁶

In contrast, OPN is ubiquitously expressed with a molecular weight of about 44 kDa.¹⁷ OPN is commonly synthesized and concentrated in bone and epithelial tissues, but has also been shown to be synthesized in the thick ascending limb and by T-cells.⁸ OPN may serve as a regulator in a number of metabolic and inflammatory diseases.⁸ In the kidney, OPN expression is upregulated in injury and recovery processes.¹⁸⁻²⁰ OPN have been shown to have protective effects on kidney function and long-term outcomes, as it is protective against nephrocalcinosis and vascular calcifications.^{21,22}

In one aspect, the present invention provides compositions and methods for measuring UMOD and/or OPN. The measured proteins can be detected as being increased or decreased relative to controls. For example, UMOD and/or OPN can be detected as being increased or decreased relative to controls. Alternatively, the detection of UMOD and OPN can be described in terms of a ratio of UMOD:OPN, above or below a particular amount being protective against graft failure.

It is understood that the ratio of UMOD to OPN may increase or decrease slightly as even more data from other cohorts is analyzed. Accordingly, in particular embodiments, the ratio of UMOD to OPN may comprise any value in between about 2 and about 4. It is further understood that the ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Thus, in particular embodiments, the ratio of UMOD to OPN can range from about 2 to about 4. In more particular embodiments, a ratio of UMOD to OPN of ≤2.0, ≤2.1, ≤2.2, ≤2.3, ≤2.4, ≤2.5, ≤2.6, ≤2.7, ≤2.8, ≤2.9, ≤3.0, ≤3.1, ≤3.2, ≤3.3, ≤3.4, ≤3.5, ≤3.6, ≤3.7, ≤3.8, ≤3.9 or ≤4.0 indicates the kidney is viable for transplantation. Consequently, a ratio of UMOD to OPN greater than the recited value indicates the kidney is not viable for transplantation. For example a ratio of UMOD to OPN of ≤3 indicates the kidney is viable for transplantation, while a ratio of UMOD to OPN above that value (i.e., >3) indicates the kidney is not viable for transplantation. Moreover, a ratio range of about 2 to about 4 includes nested ranges including, but not limited to, 2.1 to 3.9, 2.2 to 3.8, 2.3 to 3.7, 2.4 to 3.6, 2.5 to 3.5, 2.6 to 3.4, 2.7 to 3.3, 2.8 to 3.2, 2.9 to 3.1 and so forth.

### I. Definitions

“Sample” is used herein in its broadest sense. The term “biological sample” as used herein denotes a sample taken or isolated from a biological organism. A sample or biological sample may comprise a bodily fluid including urine, blood, serum, plasma, tears, aqueous and vitreous humor, spinal fluid; a soluble fraction of a cell or tissue preparation, or media in which cells were grown; or membrane isolated or extracted from a cell or tissue; polypeptides, or peptides in solution or bound to a substrate; a cell; a tissue, a tissue print, a fingerprint, skin or hair; fragments and derivatives thereof. Non-limiting examples of samples or biological samples include cheek swab; mucus; whole blood, blood, serum; plasma; urine; saliva, semen; lymph; fecal extract; sputum; other body fluid or biofluid; cell sample; and tissue sample etc. The term also includes a mixture of the above-mentioned samples or biological samples. The term “sample” also includes untreated or pretreated (or pre-processed) biological samples. In some embodiments, a sample or biological sample can comprise one or more cells from the subject. Subject samples or biological samples usually comprise derivatives of blood products, including blood, plasma and serum. In some embodiments, the sample is a biological sample. In some embodiments, the sample is blood. In some embodiments, the sample is plasma. In some embodiments, the sample is blood, plasma, serum, or urine. In certain embodiments, the sample is a urine sample.

The terms “body fluid” or “bodily fluids” are liquids originating from inside the bodies of organisms. Bodily fluids include amniotic fluid, aqueous humour, vitreous humour, bile, blood (e.g., serum), breast milk, cerebrospinal fluid, cerumen (earwax), chyle, chyme, endolymph and perilymph, exudates, feces, female ejaculate, gastric acid, gastric juice, lymph, mucus (e.g., nasal drainage and phlegm), pericardial fluid, peritoneal fluid, pleural fluid, pus, rheum, saliva, sebum (skin oil), serous fluid, semen, sputum, synovial fluid, sweat, tears, urine, vaginal secretion, and vomit. Extracellular bodily fluids include intravascular fluid (blood plasma), interstitial fluids, lymphatic fluid and transcellular fluid. “Biological sample” also includes a mixture of the above-mentioned body fluids. “Biological samples” may be untreated or pretreated (or pre-processed) biological samples.

Sample collection procedures and devices known in the art are suitable for use with various embodiment of the present invention. Examples of sample collection procedures and devices include but are not limited to: phlebotomy tubes (e.g., a vacutainer blood/specimen collection device for collection and/or storage of the blood/specimen), dried blood spots, Microvette CB300 Capillary Collection Device (Sarstedt), HemaXis blood collection devices (microfluidic technology, Hemaxis), Volumetric Absorptive Microsampling (such as CE-IVD Mitra microsampling device for accurate dried blood sampling (Neoteryx), HemaSpot™-HF Blood Collection Device, a tissue sample collection device; standard collection/storage device (e.g., a collection/storage device for collection and/or storage of a sample (e.g., blood, plasma, serum, urine, etc.); a dried blood spot sampling device. In some embodiments, the Volumetric Absorptive Microsampling (VAMS^{IM}) samples can be stored and mailed, and an assay can be performed remotely.

“Antibody” refers to a polypeptide substantially encoded by an immunoglobulin gene or immunoglobulin genes, or fragments thereof, which specifically binds and recognizes an epitope (e.g., an antigen). Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity).

The term “threshold” as used herein refers to the magnitude or intensity that must be exceeded for a certain reaction, phenomenon, result, or condition to occur or be considered relevant. The relevance can depend on context, e.g., it may refer to a positive, reactive or statistically significant relevance.

By “binding assay” is meant a biochemical assay wherein the target proteins are detected by binding to an agent, such as an antibody, through which the detection process is carried out. The detection process may involve radioactive or fluorescent labels, and the like. The assay may involve immobilization of the target protein, or may take place in solution.

“Immunoassay” is an assay that uses an antibody to specifically bind an antigen (e.g., a marker). The immunoassay is characterized by the use of specific binding properties of a particular antibody to isolate, target, and/or quantify the antigen. Non-limiting examples of immunoassays include ELISA (enzyme-linked immunosorbent assay), immunoprecipitation, SISCAPA (stable isotope standards and capture by anti-peptide antibodies), Western blot, etc.

The term “statistically significant” or “significantly” refers to statistical evidence that there is a difference. It is defined as the probability of making a decision to reject the null hypothesis when the null hypothesis is actually true. The decision is often made using the p-value.

“Detectable label” or a “label” refers to a composition detectable by electrochemiluminescent, spectroscopic, photochemical, biochemical, immunochemical, chemical or visual means. For example, useful labels include ³²P, ³⁵S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, flow cytometry, or direct analysis by mass spectrometry of intact protein or peptides. In some embodiments, the detectable moiety is a stable isotope. In some embodiments, the stable isotope is selected from the group consisting of ¹⁵N, ¹³C, ¹⁸O and ²H.

### II. Detection of Target Proteins

In specific embodiments, the UMOD and OPN target proteins of the present invention can be detected and/or measured by immunoassay. In further embodiments, YKL-40 is also detected and/or measured. Immunoassay requires biospecific capture reagents/binding agent, such as antibodies, to capture the target proteins. Many antibodies are available commercially. Antibodies also can be produced by methods well known in the art, e.g., by immunizing animals with the target proteins.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the target protein is attached to the surface of an MS probe, such as a pre-activated protein chip array. The target protein is then specifically captured on the biochip through this reagent, and the captured protein is detected by mass spectrometry.

In certain embodiments, the expression levels of the target proteins employed herein are quantified by immunoassay, such as enzyme-linked immunoassay (ELISA) technology. In specific embodiments, the levels of expression of the target proteins are determined by contacting the biological sample with antibodies, or antigen binding fragments thereof, that selectively bind to the target proteins; and detecting binding of the antibodies, or antigen binding fragments thereof, to the target proteins. In certain embodiments, the binding agents employed in the disclosed methods and compositions are labeled with a detectable moiety. In other embodiments, a binding agent and a detection agent are used, in which the detection agent is labeled with a detectable moiety.

For example, the level of a target protein(s) in a sample can be assayed by contacting the biological sample with an antibody, or antigen binding fragment thereof, that selectively binds to the target protein (referred to as a capture molecule or antibody or a binding agent), and detecting the binding of the antibody, or antigen-binding fragment thereof, to the target protein. In one embodiment, the detection can be performed using a second antibody to bind to the capture antibody complexed with its target protein. A target can be an entire protein, or a variant or modified form thereof. Kits for the detection of target proteins as described herein can include pre-coated strip/plates, biotinylated secondary antibody, standards, controls, buffers, streptavidin-horse radish peroxidase (HRP), tetramethyl benzidine (TMB), stop reagents, and detailed instructions for carrying out the tests including performing standards.

The present disclosure also provides methods for detecting target proteins such as UMOD and/or OPN in a sample obtained from a deceased donor, wherein the levels of expression of the target proteins in the sample are determined simultaneously. For example, in one embodiment, methods are provided that comprise: (a) contacting a biological sample obtained from the subject with a plurality of binding agents that each selectively bind to UMOD and OPN for a period of time sufficient to form binding agent-target protein complexes; and (b) detecting binding of the binding agents to the target proteins. In further embodiments, detection thereby determines the levels of expression of the target proteins in the biological sample; and the method can further comprise (c) comparing the levels of expression of the UMOD and/or OPN proteins in the biological sample with predetermined threshold values, wherein levels of expression of UMOD and/or OPN proteins above or below the predetermined threshold values indicates, for example, the donor's kidney is viable for transplantation. Alternatively, the ratio of UMOD to OPN can be used to indicate whether a deceased donor's kidney is viable for transplantation. In another embodiment, YKL-40 is also detected. Examples of binding agents that can be effectively employed in such methods include, but are not limited to, antibodies or antigen-binding fragments thereof, aptamers, lectins and the like.

Although antibodies are useful because of their extensive characterization, any other suitable agent (e.g., a peptide, an aptamer, or a small organic molecule) that specifically binds a target protein of the present invention is optionally used in place of the antibody in the above described immunoassays. For example, an aptamer that specifically binds a target protein and/or one or more of its breakdown products might be used. Aptamers are nucleic acid-based molecules that bind specific ligands. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

In one method of the present invention, a first capture or binding agent, such as an antibody that specifically binds UMOD, is immobilized on a suitable solid phase substrate or carrier. The test biological sample is then contacted with the capture antibody and incubated for a desired period of time. After washing to remove unbound material, a second antibody (detection) that binds to a different, non-overlapping, epitope on the target protein (or to the bound capture antibody) is then used to detect binding of the polypeptide target to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

In another embodiment, the assay is a competitive binding assay, wherein labeled UMOD target protein is used in place of the labeled detection antibody, and the labeled UMOD and any unlabeled UMOD present in the test sample compete for binding to the capture antibody. The amount of UMOD target protein bound to the capture antibody can be determined based on the proportion of labeled UMOD target protein detected.

Solid phase substrates, or carriers, that can be effectively employed in such assays are well known to those of skill in the art and include, for example, 96 well microtiter plates, glass, paper, and microporous membranes constructed, for example, of nitrocellulose, nylon, polyvinylidene difluoride, polyester, cellulose acetate, mixed cellulose esters and polycarbonate. Suitable microporous membranes include, for example, those described in US Patent Application Publication no. US 2010/0093557 A1. Methods for the automation of immunoassays are well known in the art and include, for example, those described in U.S. Pat. Nos. 5,885,530, 4,981,785, 6,159,750 and 5,358,691.

The presence of target proteins such as UMOD and OPN, as well as YKL-40, in a test sample can be detected simultaneously using a multiplex assay, such as a multiplex ELISA. Multiplex assays offer the advantages of high throughput, a small volume of sample being required, and the ability to detect different proteins across a board dynamic range of concentrations.

In certain embodiments, such methods employ an array, wherein multiple binding agents (for example capture antibodies) specific for multiple target proteins are immobilized on a substrate, such as a membrane, with each capture agent being positioned at a specific, pre-determined, location on the substrate. Methods for performing assays employing such arrays include those described, for example, in US Patent Application Publication nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminesence technology, can be used. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-target protein complexes formed on the bead set. Multiple target proteins can be recognized and measured by differences in the bead sets, with chromogenic or fluorogenic emissions being detected using flow cytometric analysis.

In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple target proteins at the corresponding spots on the plate.

In several embodiments, the target proteins of the present invention including UMOD, OPN and YKL-40, may be detected by means of an electrochemicaluminescent assay including, but not limited to, the ECL assay developed by Meso Scale Discovery (Gaithersrburg, Md.). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. Nos. 7,497,997; 7,491,540; 7,288,410; 7,036,946; 7,052,861; 6,977,722; 6,919,173; 6,673,533; 6,413,783; 6,362,011; 6,319,670; 6,207,369; 6,140,045; 6,090,545; and 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

The target proteins of the present invention can be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, Calif.), Invitrogen Corp. (Carlsbad, Calif.), Affymetrix, Inc. (Fremong, Calif.), Zyomyx (Hayward, Calif.), R&D Systems, Inc. (Minneapolis, Minn.), Biacore (Uppsala, Sweden) and Procognia (Berkshire, UK). Examples of such protein biochips are described in the following patents or published patent applications: U.S. Pat. Nos. 6,537,749; 6,329,209; 6,225,047; 5,242,828; PCT International Publication No. WO 00/56934; and PCT International Publication No. WO 03/048768.

In a particular embodiment, the present invention comprises a microarray chip. More specifically, the chip comprises a small wafer that carries a collection of binding agents bound to its surface in an orderly pattern, each binding agent occupying a specific position on the chip. The set of binding agents specifically bind to each of the target proteins described herein. In particular embodiments, a few microliters of a biological sample are dropped on the chip array. The target proteins present in the tested specimen bind to the binding agents specifically recognized by them. Subtype and amount of bound target protein is detected and quantified using, for example, a fluorescently-labeled secondary, subtype-specific antibody. In particular embodiments, an optical reader is used for bound target protein detection and quantification. Thus, a system can comprise a chip array and an optical reader. In other embodiments, a chip is provided.

### III. Point-Of-Care Assays for Detecting Target Proteins Including Uromodulin, Osteopontin and YKL-40

The types of assays described above are amenable to developing point-of-care (POC) devices, in which systems can be self-contained so that output is readable by the user. This characteristic is especially useful when collection of a sample to be tested does not require medical intervention (e.g., urine, saliva, or sputum). One device that enables this is the lateral-flow device (LFD). These devices use a multi-layered construction containing both absorbent and non-absorbent components to form a solid-phase. The capture and/or recognition reagents (antigen or antibody) are pre-applied to specific areas within the assembled apparatus and the analyte is allowed to flow through the system to come into contact with reagents. Often, for the purpose of self-containment, the reagent components are added in a dried state so that fluid from the sample re-hydrates and activates them. Conventional ELISA techniques can then be used to detect the analyte in the antigen-antibody complex. In some embodiments, the system can be designed to provide a colorimetric reading for visual estimation of a binary response (‘yes’ or ‘no’), or it can be configured to be quantitative.

In certain embodiments, the presently disclosed methods can use a lateral flow device or dipstick assay comprising an immunochromatographic strip test that relies on a direct (double antibody sandwich) reaction. Without wishing to be bound to any one particular theory, this direct reaction scheme can be used when sampling for larger analytes that may have multiple antigenic sites. Different antibody combinations can be used, for example different antibodies can be included on the capture (detection) line, the control line, and included in the mobile phase of the assay, for example, as conjugated to gold particles, e.g., gold microparticles, gold nanoparticles, or fluorescent dyes.

The term “dipstick assay” as used herein means any assay using a dipstick in which sample solution is contacted with the dipstick to cause sample solution to move by capillary action to a capture zone of the dipstick thereby allowing a target antigen in the sample solution to be captured and detected at the capture zone. To test for the presence of analyte, the contact end of the dipstick is contacted with the test solution. If analyte is present in the test solution it travels to the capture zone of the dipstick by capillary action where it is captured by the capture antibody. The presence of analyte at the capture zone of the dipstick is detected by a further anti-analyte antibody (the detection antibody) labelled with, for example, colloidal gold.

These dipstick tests have several advantages. They are easy and cheap to perform, no specialist instruments are required, and the results are obtained rapidly and can be read visually. These tests are, therefore, particularly suited for use in a physician's office, at home, in remote areas, and in developing countries where specialist equipment may not be available. They can be used, for example, to test whether a donor's kidney is viable for transplantation.

To perform a method of the first aspect of the invention, the targeting agent and labels may simply be added to the test solution and the test solution then contacted with the contact end of the chromatographic strip. Such methods are easier to perform than the method disclosed in WO 00/25135 in which two separate wicking steps are required. The results may, therefore, be obtained more rapidly, and yet the sensitivity of analyte detection is higher.

The term “chromatographic strip” is used herein to mean any porous strip of material capable of transporting a solution by capillary action. The chromatographic strip may be capable of bibulous or non-bibulous lateral flow, but preferably bibulous lateral flow. By the term “non-bibulous lateral flow” is meant liquid flow in which all of the dissolved or dispersed components of the liquid are carried at substantially equal rates and with relatively unimpaired flow laterally through the membrane as opposed to preferential retention of one or more components as would occur with “bibulous lateral flow.” Materials capable of bibulous lateral flow include paper, nitrocellulose, and nylon. A preferred example is nitrocellulose.

The labels may be bound to the targeting agent by pre-mixing the targeting agent with the labels before the targeting agent is added to (or otherwise contacted with) the test solution. However, in some circumstances, it is preferred that the targeting agent and labels are not pre-mixed because such pre-mixing can cause the targeting agent and labels to precipitate. Thus, the targeting agent and the labels may be added separately to (or contacted separately with) the test solution. The targeting agent and the labels can be added to (or contacted with) the test solution at substantially the same time, or in any order.

The test solution may be pre-incubated with the targeting agent and labels before the test solution is contacted with the contact end of the chromatographic strip to ensure complex formation. The optimal time of pre-incubation will depend on the ratio of the reagents and the flow rate of the chromatographic strip. In some cases, pre-incubation for too long can decrease the detection signal obtained, and even lead to false positive detection signals. Thus, it may be necessary to optimize the pre-incubation time for the particular conditions used.

It may be desired to pre-incubate the targeting agent with the test solution before binding the labels to the targeting agent so that the targeting agent can be allowed to bind to analyte in the test solution under optimum binding conditions.

As used herein the term “lateral flow” refers to liquid flow along the plane of a substrate or carrier, e.g., a lateral flow membrane. In general, lateral flow devices comprise a strip (or a plurality of strips in fluid communication) of material capable of transporting a solution by capillary action, i.e., a wicking or chromatographic action, wherein different areas or zones in the strip(s) contain assay reagents, which are either diffusively or non-diffusively bound to the substrate, that produce a detectable signal as the solution is transported to or migrates through such zones. Typically, such assays comprise an application zone adapted to receive a liquid sample, a reagent zone spaced laterally from and in fluid communication with the application zone, and a detection zone spaced laterally from and in fluid communication with the reagent zone. The reagent zone can comprise a compound that is mobile in the liquid and capable of interacting with an analyte in the sample, e.g., to form an analyte-reagent complex, and/or with a molecule bound in the detection zone. The detection zone may comprise a binding molecule that is immobilized on the strip and is capable of interacting with the analyte and/or the reagent and/or an analyte-reagent complex to produce a detectable signal. Such assays can be used to detect an analyte in a sample through direct (sandwich assay) or competitive binding. Examples of lateral flow devices are provided in U.S. Pat. No. 6,194,220 to Malick et al., U.S. Pat. No. 5,998,221 to Malick et al, U.S. Pat. No. 5,798,273 to Shuler et al; and U.S. Pat. No. RE38,430 to Rosenstein.

In some embodiments, the presently disclosed methods can be used with an assay comprising a sandwich lateral flow or dipstick assay. In a sandwich assay, a liquid sample that may or may not contain an analyte of interest is applied to the application zone and allowed to pass into the reagent zone by capillary action. The term “analyte” as used herein refers to a target proteins including, but not limited to UMOD, OPN and/or YKL-40. In certain embodiments the presence or absence of an analyte in a sample is determined qualitatively. In other embodiments, a quantitative determination of the amount or concentration of analyte in the sample is determined.

The analyte, if present, interacts with a labeled reagent in the reagent zone to form an analyte-reagent complex and the analyte-reagent complex moves by capillary action to the detection zone. The analyte-reagent complex becomes trapped in the detection zone by interacting with a binding molecule specific for the analyte and/or reagent. Unbound sample can pass through the detection zone by capillary action to a control zone or an absorbent pad laterally juxtaposed and in fluid communication with the detection zone. The labeled reagent may then be detected in the detection zone by appropriate means.

Generally, and without limitation, lateral flow devices comprise a sample pad. A sample pad comprises a membrane surface, also referred to herein as a “sample application zone,” adapted to receive a liquid sample. A standard cellulose sample pad has been shown to facilitate absorption and flow of biological samples, including, but not limited to, urine. The sample pad comprises a portion of lateral flow device that is in direct contact with the liquid sample, that is, it receives the sample to be tested for the analyte of interest. The sample pad can be part of, or separate from, a lateral flow membrane. Accordingly, the liquid sample can migrate, through lateral or capillary flow, from sample pad toward a portion of the lateral flow membrane comprising a detection zone. The sample pad is in fluid communication with the lateral flow membrane comprising an analyte detection zone. This fluid communication can arise through or be an overlap, top-to-bottom, or an end-to-end fluid connection between the sample pad and a lateral flow membrane. In certain embodiments, the sample pad comprises a porous material, for example and not limited to, paper.

Typically, a sample pad is positioned adjacent to and in fluid communication with a conjugate pad. A conjugate pad comprises a labeled reagent having specificity for one or more analytes of interest. In some embodiments, the conjugate pad comprises a non-absorbent, synthetic material (e.g., polyester) to ensure release of its contents. A detection conjugate is dried into place on the conjugate pad and only released when the liquid sample is applied to the sample pad. Detection conjugate can be added to the pad by immersion or spraying.

In particular embodiments, the detection conjugate comprises an antibody that specifically binds UMOD, an antibody that specifically binds OPN and/or an antibody that specifically binds YKL-40. In some embodiments, the antibody is a monoclonal antibody. In representative embodiments, the anti-UMOD antibody comprises an antibody from LSBio (Seattle, Wash.) (LS-B3105, LS-B2887, LS-C62644); Novus Biologicals, LLC (Centennial, Colo.) (NBP1-50321, Clone 10.32); or R&D Systems (Minneapolis, Minn.) (MAB5144), or combinations thereof. In other representative embodiments, the anti-OPN antibody is from Invitrogen (Carlsbad, Calif.) (MA5-17180, MA5-31217, MA5-29580); LSBio (Seattle, Wash.) (LS-B8326-100, LS-C305907-100, LS-C305911); or R&D Systems (Minneapolis, Minn.) (MAB14331), or combinations thereof. In certain representative embodiments, the YKL-40 antibody is from Millipore Sigma (Burlington, Mass.) (MABC196); Hycult Biotech, Inc. (Wayne Pa.) (HM2293); or Creative Diagnostics (Shirley, N.Y.) (clone 5924, DMAB5637MH; clone NN1739-0Y35, DCABH-3160).

The antibody, e.g., a monoclonal antibody (MAb), can be conjugated to a fluorescent dye or gold particle, e.g., colloidal gold, including gold microspheres or gold nanoparticles, such as gold nanoparticles of about 40 nm. For example, it is possible to biotinylate the conjugated MAb to take advantage of the strong affinity that biotin has for streptavidin, using Streptavidin-coated microspheres. Alternatives include protein A-coated microspheres that bind to Fc region of IgGs.

In certain embodiments, the conjugate pad is adjacent to and in fluid communication with a lateral flow membrane. Capillary action draws a fluid mixture up the sample pad, through the conjugate pad where an antibody-antigen complex is formed, and into the lateral flow membrane. Lateral flow is a function of the properties of the lateral flow membrane. The lateral flow membrane typically is extremely thin and is hydrophilic enough to be wetted, thereby permitting unimpeded lateral flow and mixture of reactants and analytes at essentially the same rates.

Lateral flow membranes can comprise any substrate capable of providing liquid flow including, but not limited to, substrates, such as nitrocellulose, nitrocellulose blends with polyester or cellulose, untreated paper, porous paper, rayon, glass fiber, acrylonitrile copolymer, plastic, glass, or nylon. Lateral flow membranes can be porous. Typically, the pores of a lateral flow membrane are of sufficient size such that particles, e.g., microparticles comprising a reagent capable of forming a complex with an analyte, flow through the entirety of the membrane. Lateral flow membranes, in general, can have a pore size ranging from about 3 μm to about 100 μm, and, in some embodiments, have a pore size ranging from about 10 μm to about 50 μm. Pore size affects capillary flow rate and the overall performance of the device.

There are multiple benefits to using nitrocellulose for the primary membrane: low cost, capillary flow, high affinity for protein biding, and ease of handlisssssssng. Nitrocellulose has high protein binding. Another alternative is cellulose acetate, which has low protein binding. Size dictating surface area dictates membrane capacity (the volume of sample that can pass through the membrane per unit time=length×width×thickness×porosity. Because these variables control the rate at which lateral flow occurs, they can impact sensitivity and specificity of the assay. The flow rate also varies with sample viscosity. Several different sizes and polymers are available for use as microspheres, which migrate down the membrane with introduction of the fluidic sample. The optimal flow rate generally is achieved using spheres that are 1/10 the pore size of the membrane or smaller.

One skilled in the art will be aware of other materials that allow liquid flow. Lateral flow membranes, in some embodiments, can comprise one or more substrates in fluid communication. For example, a conjugate pad can be present on the same substrate or may be present on separate substrates (i.e., pads) within or in fluid communication with lateral flow membranes. In some embodiments, the nitrocellulose membrane can comprise a very thin Mylar sheet coated with a nitrocellulose layer.

Lateral flow membranes can further comprise at least one indicator zone or detection zone. The terms “indicator zone” and “detection zone” are used interchangeably herein and mean the portion of the carrier or porous membrane comprising an immobilized binding reagent. As used herein, the term “binding reagent” means any molecule or a molecule bound to a particle, wherein the molecule recognizes or binds the analyte in question. The binding reagent is capable of forming a binding complex with the analyte-labeled reagent complex. The binding reagent is immobilized in the detection zone and is not affected by the lateral flow of the liquid sample due to the immobilization on the membrane. Once the binding reagent binds the analyte-labeled reagent complex it prevents the analyte-labeled reagent complex from continuing with the flow of the liquid sample. In some embodiments, the binding reagent comprises an antibody that specifically binds UMOD and an antibody that specifically binds OPN. In other embodiments, the binding reagent further comprises an antibody that binds YKL-40.

Accordingly, during the actual reaction between the analyte and the reagent, the first member binds in the indicator zone to the second member and the resulting bound complex is detected with specific antibodies. Detection may use any of a variety of labels and/or markers, e.g., enzymes (alkaline phosphatase or horseradish peroxidase with appropriate substrates), radioisotopes, liposomes or latex beads impregnated with fluorescent tags, polymer dyes or colored particles, and the like. Thus, the result can be interpreted by any direct or indirect reaction. Colloidal gold particles, which impart a purple or red coloration, are most commonly used currently.

The capture and immobilization of the assay reagent (complementary member of the binding pair) at the indicator zone can be accomplished by covalent bonding or, more commonly, by adsorption, such as by drying. Such capture also can be indirect, for example, by binding of latex beads coated with the reagent. Depending on the nature of the material comprising the lateral flow membrane, covalent bonding may be enabled, for example with use of glutaraldehyde or a carbodiimide. In immunoassays, most common binding pairs are antigen-antibody pairs; however, multiple other binding pairs can be performed, such as enzyme-substrate and receptor-ligand.

In some embodiments, the indicator zone further comprises a test line and a control line. A test line can comprise an immobilized binding reagent. When antibodies are used to develop a test line in the LFD that employs a sandwich type of assay, they are applied at a ratio of about 1-3 μg/cm across the width of a strip 1 mm wide; hence, antibody concentration is about 10-30 μg/cm², which is about 25-100 fold that used in an ELISA. Brown, M. C, Antibodies: key to a robust lateral flow immunoassay, in Lateral Flow Immunoassay, H. Y. T. R. C. Wong, Editor. 2009, Humana Press: New York, N.Y. p. 59-74.

Further, in some embodiments, the presently disclosed lateral flow assays can be used to detect multiple analytes in a sample. For example, in a lateral flow assay, the reagent zone can comprise multiple labeled reagents, each capable of binding to a different analyte in a liquid sample or a single labeled reagent capable of binding to multiple analytes. If multiple labeled reagents are used in a lateral flow assay, the reagents may be differentially labeled to distinguish different types of analytes in a liquid sample. It also is possible to place multiple lines of capture antibodies on the membrane to detect different analytes. Combinations of antibodies that detect different epitopes of an analyte may optimize specificity.

For quality control, typically a lateral flow membrane can include a control zone comprising a control line. The term “control zone” refers to a portion of the test device comprising a binding molecule configured to capture the labeled reagent. In a lateral flow assay, the control zone may be in liquid flow contact with the detection zone of the carrier, such that the labeled reagent is captured on the control line as the liquid sample is transported out of the detection zone by capillary action. Detection of the labeled reagent on the control line confirms that the assay is functioning for its intended purpose. Placement of a control line can be accomplished using a microprocessor controlled TLC spotter, in which a dispenser pump releases a constant volume of reagent across the membrane.

A typical lateral flow device can also comprises an absorbent pad. The absorbent pad comprises an “absorbent material,” which as used herein, refers to a porous material having an absorbing capacity sufficient to absorb substantially all the liquids of the assay reagents and any wash solutions and, optionally, to initiate capillary action and draw the assay liquids through the test device. Suitable absorbent materials include, for example, nitrocellulose, nitrocellulose blends with polyester or cellulose, untreated paper, porous paper, rayon, glass fiber, acrylonitrile copolymer, plastic, glass, or nylon.

In some embodiments, a lateral flow membrane is bound to one or more substantially fluid-impervious sheets, one on either side, e.g., a bottom sheet and a complimentary top sheet with one or more windows defining an application zone and an indicator zone. A typical lateral flow device also can include a housing. The term “housing” refers to any suitable enclosure for the presently disclosed lateral flow devices. Exemplary housings will be known to those skilled in the art. The housing can have, for example, a base portion and a lid portion. The lid portion can include a top wall and a substantially vertical side wall. A rim may project upwardly from the top wall and may further define a recess adapted to collect a sample from a subject. Suitable housings include those provided in U.S. Pat. No. 7,052,831 to Fletcher et al and those used in the BD Directigen™ EZ RSV lateral flow assay device.

In some embodiments, target proteins such as UMOD, OPN and/or YKL-40 can be measured in whole, unconcentrated, or otherwise unprocessed, biological samples using the presently disclosed methods and devices. In other embodiments, the biological sample can be processed, e.g., concentrated, diluted, filtered, and the like, prior to performing the test. The pre-treatment of a urine sample can include diluting the urine sample in an aqueous solution, concentrating the urine sample, filtering the urine sample, or a combination thereof.

One of ordinary skill in the art upon review of the presently disclosed subject matter would appreciate that the pre-treatment steps can be performed in any particular order, e.g., in some embodiments, the sample can be diluted or concentrated and then filtered, whereas in other embodiments, the sample can be filtered and then diluted or concentrated. In particular embodiments, the presently disclosed methods include filtering the urine sample, for example, through a desalting column, to remove a molecule that might interfere with the detection of antigen in the urine sample. This step can be performed with or without any further dilution or concentration of the sample.

Thus, in some embodiments, the lateral flow device further comprises an apparatus adapted to pre-treat the biological sample before contacting the biological sample with at least one antibody specific for UMOD and/or at least one antibody specific for OPN. In particular embodiments, the apparatus is adapted to filter, dilute, or concentrate the biological sample, or combinations thereof. In an alternative embodiment, the apparatus can be adapted to remove an inhibitor that interferes with the detection of UMOD and/or OPN in the biological sample, in particular, a urine sample.

In other embodiments, different parameters of the test, e.g., incubation time, can be manipulated to increase sensitivity and/or specificity of the test to eliminate the need for processing the biological sample.

### IV. Kits for Detecting Target Proteins Including Uromodulin, Osteopontin and YKL-40

In another aspect, the present invention provides kits for detecting target proteins including, but not limited to UMOD, OPN and YKL-40. The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

In various embodiments, the present invention provides a kit comprising: (a) one or more internal standards suitable for measurement of UMOD and/or OPN including any one or more of mass spectrometry, antibody method, antibodies, nucleic acid aptamer method, nucleic acid aptamers, immunoassay, ELISA, immunoprecipitation, SISCAPA, Western blot, or combinations thereof; and (b) reagents and instructions for sample processing, preparation and UMOD and/or OPN measurement/detection. The kit can further comprise (c) instructions for using the kit to measure the proteins in a sample obtained from the subject. The kit can further comprise an internal standard, as well as reagents and instructions for processing, preparing and measuring/detecting YKL-40.

In particular embodiments, the kit comprises reagents necessary for processing of samples and performance of an immunoassay. In a specific embodiment, the immunoassay is an ELISA. Thus, in certain embodiments, the kit comprises a substrate for performing the assay (e.g., a 96-well polystyrene plate). The substrate can be coated with antibodies specific for a target protein(s) including UMOD, OPN and YKL-40. In a further embodiment, the kit can comprise a detection antibody(ies) including, for example, a polyclonal antibody specific for a target protein conjugated to a detectable moiety or label (e.g., horseradish peroxidase). The kit can also comprise a standard, e.g., a human UMOD, OPN and/or YKL-40 standard. The kit can also comprise one or more of a buffer diluent, calibrator diluent, wash buffer concentrate, color reagent, stop solution and plate sealers (e.g., adhesive strip).

In particular embodiments, the kit may comprise a solid support, such as a chip, microtiter plate (e.g., a 96-well plate), bead, resin, membrane, dipstick, filter, or quantum dot having UMOD, OPN and YKL-40 protein capture reagents attached thereon. The kit may further comprise a means for detecting the target protein such as antibodies, and a secondary antibody-signal complex such as horseradish peroxidase (HRP)-conjugated goat anti-rabbit IgG antibody and tetramethyl benzidine (TMB) as a substrate for HRP.

The kit may be provided as an immuno-chromatography strip comprising a membrane on which the anti-UMOD, anti-OPN and/or YKL-40 antibodies are immobilized, and a means for detecting, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of the sample.

In certain embodiments, a subject can be assessed by adding a biological sample (e.g., urine) from the patient to the kit and detecting target proteins conjugated with antibodies, specifically, by a method which comprises the steps of: (i) collecting urine from the deceased donor; (ii) adding urine from the donor to a diagnostic kit; and, (iii) detecting the target proteins conjugated with antibodies. In other kit and diagnostic embodiments, urine will not be collected from the deceased donor (i.e., it is already collected). Urine samples can be collected from deceased donors of varying ages. Moreover, in other embodiments, the sample may comprise a serum, plasma, sweat, tissue, blood or a clinical sample.

The kit can also comprise a washing solution or instructions for making a washing solution, in which the combination of the capture reagents and the washing solution allows capture of the target proteins on the solid support for subsequent detection by, e.g., antibodies, mass spectrometry and the like. In a further embodiment, a kit can comprise instructions for suitable operational parameters in the form of a label or separate insert. For example, the instructions may inform a user about how to collect the sample, etc. In yet another embodiment, the kit can comprise one or more containers with target protein samples, to be used as standard(s) for calibration or normalization. Detection of the markers described herein may be accomplished using a lateral flow assay.

In particular embodiments, the target proteins of the present invention can be captured and concentrated using nano particles. In a specific embodiment, the proteins can be captured and concentrated using Nanotrap® technology (Ceres Nanosciences, Inc. (Manassas, Va.)). Briefly, the Nanotrap platform reduces pre-analytical variability by enabling target protein enrichment, removal of high-abundance analytes, and by preventing degradation to highly labile analytes in an innovative, one-step collection workflow. Multiple analytes sequestered from a single sample can be concentrated and eluted into small volumes to effectively amplify, up to 100-fold or greater depending on the starting sample volume (Shafagati, 2014; Shafagati, 2013; Longo, et al., 2009), resulting in substantial improvements to downstream analytical sensitivity.

In certain embodiments, the kit comprises reagents and components necessary for performing an electrochemiluminescent ELISA.

In certain embodiments, the kit comprises the use of a lateral flow apparatus, dipstick, assay stick with immunochromatographic detection display, and any such apparatus know to those skilled in the art. In certain embodiments, reagents and/or detection components may be immobilized on the apparatus itself (i.e., on the dipstick).

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: The Association of Tubular Epithelial Repair Markers in Deceased Donor Urine with Graft Outcomes

### Materials and Methods

Study Population. The Deceased Donor Study (DDS) is a multicenter, observational, cohort study of deceased donors and their corresponding kidney recipients. DDS includes deceased donors in collaboration with five organ procurement organizations (OPOs): Gift of Life Donor Program, Philadelphia, Pa.; New Jersey Sharing Network, New Providence, N.J.; Gift of Life Michigan, Ann Arbor, Mich.; New York Organ Donor Network, New York, N.Y.; and New England Organ Bank, Waltham, Mass. Donor urine samples were collected at the time of organ procurement from May 2010 to December 2013. Inclusion criteria included deceased donors at least 16 years of age with both admission and terminal serum creatinine. Donors were excluded if both kidneys were discarded or if they were missing urine samples.⁴ Clinical variables for deceased donors were abstracted from OPO charts, and data for recipients were obtained from the Organ Procurement and Transplantation Network (OPTN). The OPTN data system includes data on all donors, wait-listed candidates, and transplant recipients in the US, submitted by the members of the OPTN, and has been described elsewhere. The Health Resources and Services Administration, U.S. Department of Health and Human Services provides oversight to the activities of the OPTN contractor. The analyses are based on OPTN data as of Jul. 31, 2017, and may be subject to change due to future data submission or correction by transplant centers. The OPO scientific review committees and the institutional review boards for the participating investigators approved this study.

Operational Definitions of Outcome Variables. The primary outcomes of interest were donor AKI and death-censored graft failure (dcGF). Donor AKI was defined as a 50% increase in terminal serum creatinine concentration from admission or an absolute increase in serum creatinine of 0.3 mg/dL, irrespective of urine output or time from admission to terminal serum creatinine measurement. Stages of AKI were defined by Acute Kidney Injury Network criteria. Our secondary outcome of interest was all-cause GF, which was defined as all-cause mortality, return to dialysis, or re-transplantation.

Measurement of MOD and OPN. Upon transfer to the donor operating room, 10 ml of urine was obtained from the catheter tubing and then transported on ice to the OPO, where it was stored at −80° C. Samples were delivered to the Yale University biorepository monthly. Upon arrival to the biorepository, samples underwent a single controlled thaw, were centrifuged at 2000 g for 10 minutes at 4° C., separated into 1-ml aliquots, and immediately stored at −80° C. until UMOD and OPN measurements. Both repair markers were measured using the Meso Scale Discovery platform (Meso Scale Diagnostics, Gaithersburg, Md.), which uses electrochemiluminescence detection combined with patterned arrays. All laboratory personnel were blinded to donor and recipient information.

Statistical Analysis. Continuous variables were reported as mean (standard deviation, SD) or median (interquartile range, IQR). Categorical variables were reported as frequencies, n (%). Differences in clinical and demographic characteristics were evaluated by the Kruskal-Wallis test or Chi-square test for continuous or categorical variables, respectively. As no clinically accepted cut-offs are available for UMOD and OPN, the present inventors evaluated the associations between these two markers and outcomes both as continuous (log₂-transformed) and categorical (tertiles) variables. The present inventors used logistic regression models to evaluate the association between each repair marker and donor AKI. Our logistic model for donor AKI adjusted for the following donor characteristics: age, body mass index, black race, hypertension, diabetes, stroke as the cause of death, hepatitis C serostatus, donation after circulatory determination of death status, and terminal urine creatinine. The odds ratios and 95% confidence intervals of both the univariable and multivariable models are reported.

The present inventors used Cox proportional hazard models to assess the associations of the proteins with dcGF and GF. The proportional hazards assumption was evaluated by Kolmogorov-type supremum test. The hazard ratios and 95% confidence intervals of both the univariable and multivariable models are reported. Since one donor may have one or two recipients, the present inventors estimated 95% confidence intervals using a robust sandwich covariance matrix estimator to account for intracluster dependence.²³ All inference testing was two-sided with a significance level of 0.05. Cox proportional hazards models adjusted for kidney donor risk index (KDRI), urine creatinine, cold ischemia time, and the following recipient characteristics: age, black race, sex, previous kidney transplant, diabetes as the cause of end-stage kidney disease, number of human leukocyte antigen mismatches, panel reactive antibody, body mass index, and pre-emptive transplant.

The present inventors randomly divided our cohort of 2430 recipients into a training dataset and a test dataset with 1215 recipients and their corresponding donors in each dataset. In the training dataset, the present inventors explored combinations of UMOD and OPN and the association with dcGF. Given the opposing associations of UMOD and OPN with renal outcomes in prior literature,^{16, 22} the present inventors evaluated the ratio of UMOD to OPN continuously (ratio of log₂-transformed UMOD and log₂-transformed OPN) and as a categorical variable (tertiles of the ratio) to assess the association of the combined repair markers with dcGF and all-cause GF. Tertile categories were derived from spline plots, and a data-driven cut-point of greater than 3 was established based on the ratio values in the third tertile. Given that there is no established ratio in the literature or a clinically established cut-off, the present inventors enhanced the validity of our results by deriving univariate and multivariate Cox proportional hazards in the training data set and then internally validating our results in the test data set.

All analyses were conducted on SAS 9.4 software (SAS Institute, Cary, N.C.) and Stata version 14 (StataCorp LLC).

Immunofluorescence Staining and Quantification. The present inventors performed double staining for both UMOD and OPN on 11 deceased donor tissue samples from a pathology biobank (6 biopsies with acute tubular injury and 4 biopsies without acute tubular injury). Antigen retrieval was performed with citrate (pH 5.8) and endogenous peroxidase and alkaline phosphatase reactions were blocked with levamisole hydrochloride (abcam, Cambridge, Mass.) and PolyDetector peroxidase block (BioSB, Santa Barbara, Calif.) for 10 minutes. Tissue sections were incubated for 60 minutes with mouse monoclonal OPN antibody (1:200, LFMb-14, Santa Cruz Biotechnology, Inc., Dallas, Tex.) and rabbit polyclonal anti-UMOD antibody (1:1000, MilliporeSigma, St. Louis, Mo. Detection was performed with using horseradish peroxidase polymer anti-mouse IgG with Emerald green substrate and alkaline phosphatase polymer anti-rabbit IgG with permanent red substrate (DoubleStain IHC Kit abcam, Cambridge, Mass.). OPN was interpreted as positive in green-stained areas, while red stain indicated UMOD positivity. Co-localization was appreciated as follows: blue—OPN expressed at higher concentrations compared to UMOD; purple—UMOD was expressed at higher concentrations.

### Results

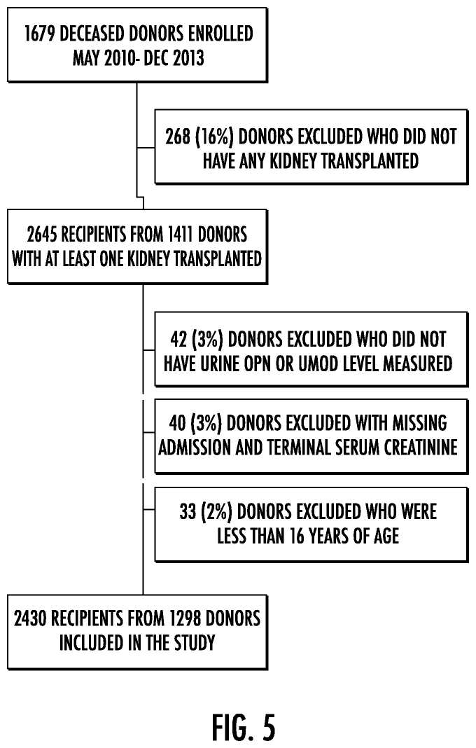

Donor and Recipient Characteristics. A total of 1298 donors and 2430 recipients met the inclusion criteria (FIG. 5). Donors had a mean (SD) age of 41 (15) years; 784 (60%) were male and 205 (16%) were black (Table 1). Recipients had a mean age of 53 (15) years; 1492 (61%) were male and 956 (39%) were black (Table 1). Donation after neurologic determination of death occurred in 1092 (94%) donors. The most frequent comorbidities among donors were hypertension (31%), diabetes (10%), and obesity (32%). For recipients, the most common causes of end-stage kidney disease were diabetes (30%) and hypertension (26%). Mean kidney donor profile index was 48 (27). Most donor and recipient characteristics were not significantly different by UMOD or OPN tertiles (Tables 5a-5b). Terminal serum creatinine, however, was greater with increasing UMOD tertiles but lower with increasing OPN tertiles. Cold ischemia time and the number of human leukocyte antigen mismatches were also greater with increasing tertiles of both UMOD and OPN.

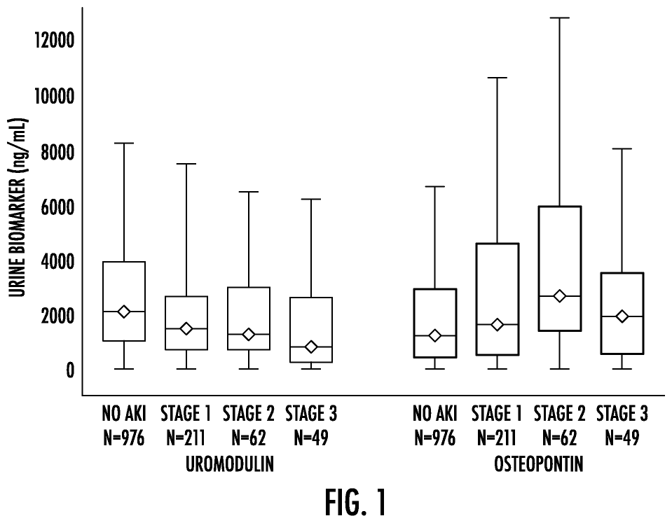

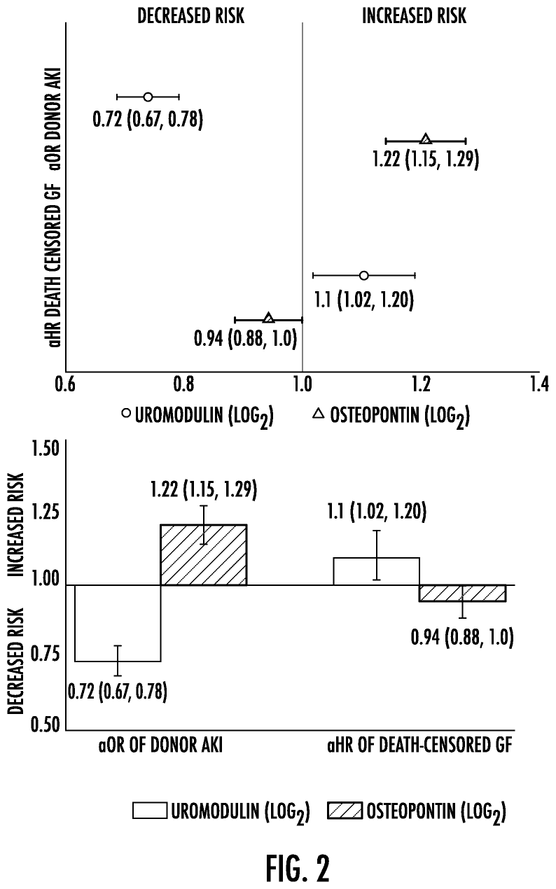

Association of UMOD and OPN with Donor AKI. A total of 322 (25%) donors had AKI (Table 1), with the majority having stage 1 AKI (16%), followed by stage 2 (5%) and stage 3 (4%). Donor urine UMOD concentrations significantly decreased with increasing AKI stage (FIG. 1). This trend remained consistent after indexing UMOD to urine creatinine (Table 7). Donor AKI was independently associated with decreased levels of urine UMOD [adjusted odds ratio, aOR (95% CI) 0.72 (0.65-0.80)] as shown in Table 2 and FIG. 2.

Levels of urine OPN increased with worsening AKI severity up to stage 2 (FIG. 1) with a consistent pattern after indexing to urine creatinine (Table 7). Donor AKI was independently associated with increased levels of urine OPN [aOR (95% CI) 1.18 (1.09-1.28)] as shown in Table 2 and FIG. 2.

Association of UMOD and OPN with Recipient Outcomes. The mean event rate (95% CI) for dcGF and GF was 33 (29.6, 36.9) and 65.7 (60.8, 71.1) per 1000-person years, respectively, over a median (IQR) follow up time of 4.01 (2.97, 5.01) years.

Each doubling of UMOD levels in donor urine was associated with increased risk for dcGF and GF in recipients with adjusted hazard ratios [aHR (95% CI)] of 1.10 (1.01-1.19) and 1.07 (1.01-1.13), respectively, after adjustment for KDRI, donor urine creatinine, and clinical covariates (Table 3). Tertiles of UMOD demonstrated increasing event rates of dcGF and GF, though HRs were not significant for UMOD tertiles. There were no significant interactions by donor AKI status on the relationship between UMOD and dcGF or GF.

Each doubling of donor urine OPN concentration was independently associated with decreased risk for dcGF [0.95 (0.89-1)] and GF [0.96 (0.93-1)]. A dose-response effect was observed such that the upper tertile showed a significant protective effect against GF compared to the lower tertile of donor urine OPN.

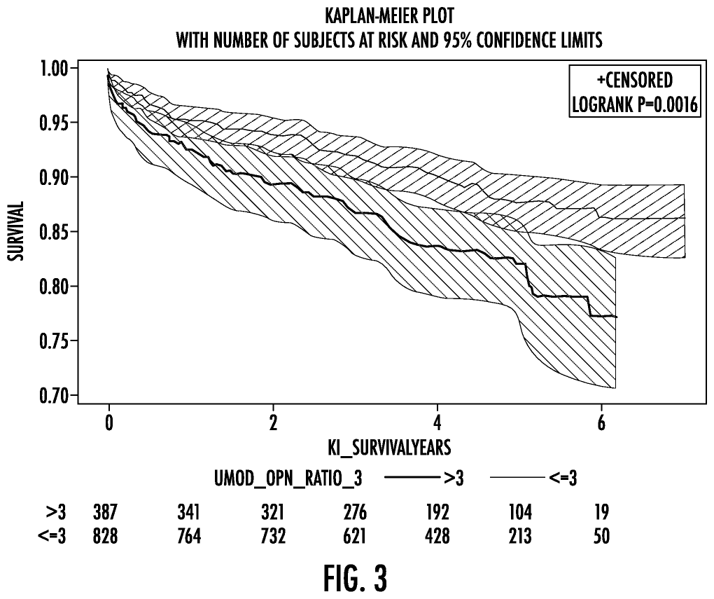

Uromodulin Osteopontin Ratio. In order to create a target protein score for clinical application, the present inventors explored various statistical combinations including the ratio of UMOD to OPN urinary levels at the time of organ procurement in our training dataset. The baseline characteristics of donors and recipients in the training and test datasets are shown in Table 8a and 8b. The ratio of UMOD to OPN demonstrated independent associations in the training dataset (Table 4). In FIG. 3, unadjusted Kaplan-Meier curves showed significantly lower graft survival when UMOD/OPN ratio was >3 as compared to ≤3 (log-rank p=0.0016). In fully adjusted models as shown in Table 4, participants with a ratio ≤3 had a 43% and 26% decreased risk of dcGF and GF, respectively. There were no significant interactions by donor AKI status on the relationship between UMOD to OPN ratio and dcGF or GF. In the test dataset, the association for GF was confirmed. The association for dcGF lost statistical significance but had a similar estimate.

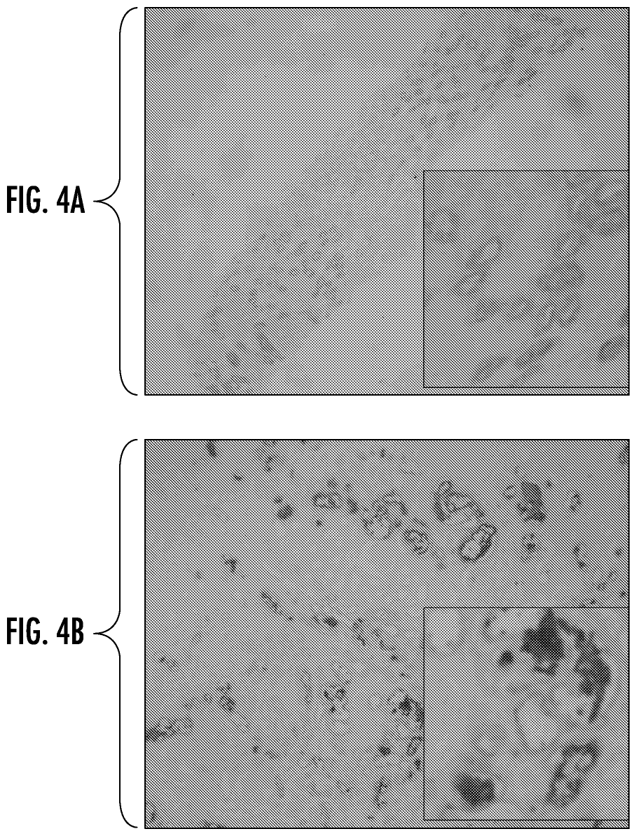

Immunofluorescence Staining and Quantification. In FIG. 4, Panel A, the dual stain for UMOD (red) and OPN (teal) shows limited staining mostly in the loop of Henle in control tissues and OPN is negative (n=4). Tubular casts and injured tubules including proximal tubules and loop of Henle stain for OPN and UMOD in deceased donor biopsies showing acute tubular injury (n=6) (FIG. 4B). Immunohistochemical assessment of deceased donor kidneys with histomorphologic evidence of acute tubular injury confirms the increased expression of OPN in injured tubular segments together with UMOD. Details on the age, sex, creatinine, and histological findings of the tissue donors can be seen in Table 9.

### DISCUSSION

In the prospective DDS cohort, donor urine UMOD levels decreased while OPN levels increased with increasing severity of donor AKI. UMOD was associated with increased risk of dcGF while OPN demonstrated a protective association with regard to dcGF. A ratio of UMOD to OPN ≤3 at the time of organ procurement was protective against dcGF and our secondary outcome of all-cause GF. The ratio of these two markers provides a construct that captures their bidirectional associations, which may help identify deceased-donor kidneys at the time of organ procurement that are more likely to have favorable outcomes than unfavorable outcomes.

In prior literature, higher levels of baseline urine UMOD have been shown to increase the risk of incident chronic kidney disease and type 1 diabetic nephropathy.^{16,24} On the other hand, OPN expression has been shown to be higher in patients with recovery from AKI²² and has also been shown to be protective against nephrocalcinosis.²¹ In our cohort, urine UMOD levels decrease below normal levels with increasing stages of donor AKI, an association that is consistent with prior AKI studies in the setting of cardiac surgery.^{25,26} Similarly, donors in our cohort with increasing stages of AKI had levels of urine OPN that exceeded levels in healthy adults established by Min et al (˜1900 ng/mL).²⁷ The positive association between OPN and AKI is consistent with prior studies.⁴⁴ The excretion patterns for these two markers with donor AKI were unchanged after indexing to urine creatinine, suggesting that these findings represent true target protein changes in urine. Staining results for UMOD and OPN among biopsies from donors with and without ATI confirm expression patterns seen in donor urine measurements.

Furthermore, the biopsy findings show that OPN increases in tandem with UMOD during donor level injury. Our population-based findings suggest that donor urine OPN was protective against graft failure while urine UMOD was associated with graft failure, without significant interactions by AKI status. Together, the balance of donor urine OPN and UMOD captured in ratio form may not only provide more granular information on graft quality than serum creatinine, but also characterize a kidney's recovery potential after fluctuations in serum creatinine (AKI) prior to nephrectomy. In our study, a UMOD OPN ratio ≤3 was protective against dcGF and GF.

There are several strengths to our study. The DDS study is a large prospective cohort that includes both donor urine measurements and recipient outcomes. This unique design allows us to investigate potential tools to improve kidney allocation decisions. In our study, the timing of our urine target protein samples coincides with trajectories established in the current literature for urine UMOD and OPN. UMOD production increases after 48 hours of ischemia-reperfusion injury.²⁸ Similarly, murine studies suggest that OPN increases after 24 hours, and continues to increase up to 5-7 days after ischemia-reperfusion injury.²⁹ Our donor urine measurements capture these target protein increases. Additionally, we accounted for differing donor urine volumes and dilution by adjusting for urine creatinine in our analyses. Finally, our ratio findings were developed in a training dataset and validated in a test dataset, which suggests that our findings were not due to resubstitution bias or model-selection bias.³⁰

There are also several limitations worth noting. First, both markers were measured at a single time point of organ procurement. As with all observational studies, our study is subject to unmeasured confounding that could have affected the identified associations. Finally, although our results were internally validated and showed statistical and clinical significance, we acknowledge that external validation will be necessary to advance these findings to clinical practice.

In conclusion, our study shows moderately strong associations of UMOD and OPN with donor and recipient outcomes. A ratio of UMOD to OPN ≤3 was protective against dcGF and GF. These findings were validated in our test dataset and are consistent with dual in deceased donor kidneys with injury. This ratio may be a clinically meaningful method for capturing the dynamic processes that take place in deceased-donor kidney transplantation and may offer a more timely and accurate way to help allocate donor kidneys than is currently available in clinical practice.

### REFERENCES

1. Schuurs T A, Morariu A M, Ottens P J, et al. Time-dependent changes in donor brain death related processes. American journal of transplantation: official journal of the American Society of Transplantation and the American Society of Transplant Surgeons. 2006; 6(12):2903-2911.
2. Devarajan P. Update on mechanisms of ischemic acute kidney injury. Journal of the American Society of Nephrology: JASN. 2006; 17(6):1503-1520.
3. Mansour S G, Puthumana J, Reese P P, et al. Associations between Deceased-Donor Urine MCP-1 and Kidney Transplant Outcomes. Kidney Int Rep. 2017; 2(4):749-758.
4. Reese P P, Hall I E, Weng F L, et al. Associations between Deceased-Donor Urine Injury Biomarkers and Kidney Transplant Outcomes. Journal of the American Society of Nephrology: JASN. 2016; 27(5):1534-1543.
5. Basile D P, Bonventre J V, Mehta R, et al. Progression after AKI: Understanding Maladaptive Repair Processes to Predict and Identify Therapeutic Treatments. Journal of the American Society of Nephrology: JASN. 2016; 27(3):687-697.
6. Puthumana J, Hall I E, Reese P P, et al. YKL-40 Associates with Renal Recovery in Deceased Donor Kidney Transplantation. Journal of the American Society of Nephrology: JASN. 2017; 28(2):661-670.
7. Cavallone D, Malagolini N, Serafini-Cessi F. Mechanism of release of urinary Tamm-Horsfall glycoprotein from the kidney GPI-anchored counterpart. Biochem Biophys Res Commun. 2001; 280(1):110-114.
8. Xie Y, Sakatsume M, Nishi S, Narita I, Arakawa M, Gejyo F. Expression, roles, receptors, and regulation of osteopontin in the kidney. Kidney Int. 2001; 60(5):1645-1657.
9. de Baaij J H, Groot Koerkamp M J, Lavrijsen M, et al. Elucidation of the distal convoluted tubule transcriptome identifies new candidate genes involved in renal Mg(2+) handling. Am J Physiol Renal Physiol. 2013; 305(11):F1563-1573.
10. Sikri K L, Foster C L, MacHugh N, Marshall R D. Localization of Tamm-Horsfall glycoprotein in the human kidney using immuno-fluorescence and immuno-electron microscopical techniques. Journal of anatomy. 1981; 132(Pt 4):597-605.
11. Bokhove M, Nishimura K, Brunati M, et al. A structured interdomain linker directs self-polymerization of human uromodulin. Proceedings of the National Academy of Sciences of the United States of America. 2016; 113(6):1552-1557.
12. Wenk R E, Bhagavan B S, Rudert J. Tamm-Horsfall uromucoprotein and the pathogenesis of casts, reflux nephropathy, and nephritides. Pathobiol Annu. 1981; 11:229-257.
13. Su S J, Chang K L, Lin T M, Huang Y H, Yeh T M. Uromodulin and Tamm-Horsfall protein induce human monocytes to secrete TNF and express tissue factor. J Immunol. 1997; 158(7):3449-3456.
14. Saemann M D, Weichhart T, Zeyda M, et al. Tamm-Horsfall glycoprotein links innate immune cell activation with adaptive immunity via a Toll-like receptor-4-dependent mechanism. The Journal of clinical investigation. 2005; 115(2):468-475.
15. Kreft B, Jabs W J, Laskay T, et al. Polarized expression of Tamm-Horsfall protein by renal tubular epithelial cells activates human granulocytes. Infect Immun. 2002; 70(5):2650-2656.
16. Kottgen A, Hwang S J, Larson M G, et al. Uromodulin levels associate with a common LTMOD variant and risk for incident CKD. Journal of the American Society of Nephrology: JASN. 2010; 21(2):337-344.
17. Kahles F, Findeisen H M, Bruemmer D. Osteopontin: A novel regulator at the cross roads of inflammation, obesity and diabetes. Mol Metab. 2014; 3(4):384-393.
18. Denhardt D T, Mistretta D, Chambers A F, et al. Transcriptional regulation of osteopontin and the metastatic phenotype: evidence for a Ras-activated enhancer in the human OPN promoter. Clin Exp Metastasis. 2003; 20(1):77-84.
19. Lund S A, Wilson C L, Raines E W, Tang J, Giachelli C M, Scatena M. Osteopontin mediates macrophage chemotaxis via alpha4 and alpha9 integrins and survival via the alpha4 integrin. J Cell Biochem. 2013; 114(5):1194-1202.
20. Lund S A, Giachelli C M, Scatena M. The role of osteopontin in inflammatory processes. J Cell Commun Signal. 2009; 3(3-4):311-322.
21. Paloian N J, Leaf E M, Giachelli C M. Osteopontin protects against high phosphate-induced nephrocalcinosis and vascular calcification. Kidney Int. 2016; 89(5):1027-1036.
22. Lorenzen J M, Hafer C, Faulhaber-Walter R, et al. Osteopontin predicts survival in critically ill patients with acute kidney injury. Nephrol Dial Transplant. 2011; 26(2):531-537.
23. Lee E. W. WLJ, Amato D. A., Leurgans S. Cox-Type Regression Analysis for Large Numbers of Small Groups of Correlated Failure Time Observations. Vol 211: Springer, Dordrecht; 1992.
24. Schlatzer D, Maahs D M, Chance M R, et al. Novel urinary protein biomarkers predicting the development of microalbuminuria and renal function decline in type 1 diabetes. Diabetes Care. 2012; 35(3):549-555.
25. Garimella P S, Jaber B L, Tighiouart H, et al. Association of Preoperative Urinary Uromodulin with AKI after Cardiac Surgery. Clinical journal of the American Society of Nephrology: CJASN. 2017; 12(1): 10-18.
26. Askenazi D J, Koralkar R, Patil N, Halloran B, Ambalavanan N, Griffin R. Acute Kidney Injury Urine Biomarkers in Very Low-Birth-Weight Infants. Clinical journal of the American Society of Nephrology: CJASN. 2016; 11 (9):1527-1535.
27. Min W, Shiraga H, Chalko C, Goldfarb S, Krishna G G, Hoyer J R. Quantitative studies of human urinary excretion of uropontin. Kidney Int. 1998; 53(1):189-193.
28. El-Achkar T M, McCracken R, Liu Y, et al. Tamm-Horsfall protein translocates to the basolateral domain of thick ascending limbs, interstitium, and circulation during recovery from acute kidney injury. Am J Physiol Renal Physiol. 2013; 304(8):F1066-1075.
29. Persy V P, Verstrepen W A, Ysebaert D K, De Greef K E, De Broe M E. Differences in osteopontin up-regulation between proximal and distal tubules after renal ischemia/reperfusion. Kidney Int. 1999; 56(2):601-611.
30. Kerr K F, Meisner A, Thiessen-Philbrook H, Coca S G, Parikh C R. Developing risk prediction models for kidney injury and assessing incremental value for novel biomarkers. Clinical journal of the American Society of Nephrology: CJASN. 2014; 9(8): 1488-1496.

TABLE 1
Donor and Recipient Characteristics in Overall Cohort

| Donor Characteristics | N = 1298 | | Recipient Characteristics | N = 2430 | |
|---|---|---|---|---|---|
| Age, years | 41.44 | (14.53) | Age, years | 52.91 | (14.83) |
| Male | 784 | (60%) | Male | 1492 | (61%) |
| Black Race | 205 | (16%) | Black Race | 956 | (39%) |
| Hispanic Race | 171 | (13%) | Hispanic race | 279 | (11%) |
| Body mass index, kg/m² | 28.42 | (7.23) | Body mass index, kg/m²) | 28.04 | (5.76) |
| Hypertension | 399 | (31%) | Cause of ESKD | | |
| Diabetes | 130 | (10%) | Unknown/other | 496 | (20%) |
| Cause of Death | | | Diabetes | 726 | (30%) |
| Head Trauma | 396 | (31%) | Hypertension | 643 | (26%) |
| Anoxia | 425 | (34%) | Glomerulonephritis | 391 | (16%) |
| Stroke | 427 | (34%) | Graft Failure | 174 | (7%) |
| Other | 18 | (1%) | ESKD duration (months) | 45.85 | (38.06) |
| Hepatitis C | 48 | (4%) | Preemptive Transplant | 274 | (11%) |
| DCD including DND | 246 | (19%) | Previous Kidney Transplant | 315 | (13%) |
| DCD | 206 | (16%) | Recipient Transfusions | 438 | (18%) |
| KDPI based on KDRI | 48.23 | (27.34) | Candidate most recent PRA | 21% | (35%) |
| Days from admission to death | 5.07 | (6.7) | Recipient panel reactive antibody | | |
| Admission SCr, mg/dL | 1.1 | (0.61) | 0% | 1545 | (64%) |
| Terminal SCr, mg/dL | 1.17 | (0.85) | 1-20% | 178 | (7%) |
| AKI Stage | | | 21-80% | 326 | (13%) |
| No AKI | 976 | (75%) | >80% | 381 | (16%) |
| Stage 1 | 211 | (16%) | Kidney Biopsied | 1117 | (46%) |
| Stage 2 | 62 | (5%) | Kidney Pumped | 952 | (39%) |
| Stage 3 | 49 | (4%) | Cold ischemia Time, hours | 15.29 | (7.1) |
| Kidneys discarded | | | HLA mismatch level | 4.21 | (1.52) |
| 0 | 1132 | (87%) | | | |
| 1 | 166 | (13%) | | | |

Values are means (SD) or n (%).
Abbreviations: AKI, acute kidney injury; BMI, body mass index; ESKD, end-stage renal disease; DCD, donation after cardiac determination of death; DND, donation after neurologic determination of death; HLA, human leukocyte antigen; KDRI, kidney donor risk index; KDPI, kidney donor profile index; PRA, panel reactive antibodies; SCr, serum creatinine.

TABLE 2
Association of Donor UMOD and OPN with Donor AKI

| | | | | | |
|---|---|---|---|---|---|
| | Range of Values | Donors with | Unadjusted OR | Model 1^{a} | Model 2^{b} |
| Urine Markers | (ng/mL) | AKI n (%) | (95% CI) | OR (95% CI) | OR (95% CI) |

Uromodulin

| | | | | | | | | | |
|---|---|---|---|---|---|---|---|---|---|
| Log₂ (n = 1298) | | 322 | (25%) | 0.77 | (0.71, 0.84) | 0.76 | (0.7, 0.84) | 0.72 | (0.65, 0.8) |
| Tertile 1 (n = 439) | (31, 1248) | 148 | (34%) | 1 | (ref) | 1 | (ref) | 1 | (ref) |
| Tertile 2 (n = 434) | (1248, 3052) | 92 | (21%) | 0.53 | (0.39, 0.72) | 0.55 | (0.4, 0.75) | 0.5 | (0.42, 0.68) |
| Tertile 3 (n = 425) | (3065, 40, 400) | 82 | (19%) | 0.47 | (0.34, 0.64) | 0.48 | (0.35, 0.67) | 0.41 | (0.29, 0.59) |

Osteopontin

| | | | | | | | | | |
|---|---|---|---|---|---|---|---|---|---|
| Log₂ (n = 1298) | | 322 | (25%) | 1.14 | (1.06, 1.22) | 1.15 | (1.07, 1.23) | 1.18 | (1.09, 1.28) |
| Tertile 1 (n = 433) | (53, 808) | 148 | (34%) | 1 | (ref) | 1 | (ref) | 1 | (ref) |
| Tertile 2 (n = 433) | (809, 2445) | 92 | (21%) | 1.1 | (0.8, 1.52) | 1.17 | (0.84, 1.63) | 1.22 | (0.87, 1.72) |
| Tertile 3 (n = 432) | (2448, 46, 100) | 82 | (19%) | 1.61 | (1.19, 2.2) | 1.71 | (1.24, 2.35) | 1.86 | (1.3, 2.67) |

^{a}Model 1 includes donor age (years), BMI, black race, history of hypertension, history of diabetes, stroke as cause of death, hepatitis C serostatus, and donation after circulatory determination of death status.
^{b}Model 2 includes donor variables from model 1 and donor urine creatinine.

TABLE 3
Association of Donor UMOD and OPN with Risk of All-
Cause Graft Failure and Death-Censored Graft Failure

| | | | Hazard Ratio (95% Confidence Interval) | | | |
|---|---|---|---|---|---|---|
| | Mean (95% CI) Event | | | | | |
| | Rate per 1000 Person | | | | | |
| | Year | | Unadjusted | | Adjusted^{a} | |
| | Death-Censored Graft Failure (dcGF) | | | | | |
| Uromodulin | | | | | | |
| Log2 (n = 2430) | 33 | (29.6, 36.9) | 1.11 | (1.03, 1.19) | 1.1 | (1.02, 1.2) |
| Tertile 1 (n = 810) | 28.6 | (23.2, 35.2) | 1 | (ref) | 1 | (ref) |
| Tertile 2 (n = 810) | 33.9 | (28.1, 41) | 1.19 | (0.9, 1.57) | 1.12 | (0.84, 1.5) |
| Tertile 3 (n = 810) | 36.4 | (30.4, 43.6) | 1.27 | (0.96, 1.67) | 1.2 | (0.89, 1.62) |
| Osteopontin | | | | | | |
| Log2 (n = 2430) | 33 | (29.6, 36.9) | 0.95 | (0.89, 1) | 0.94 | (0.88, 1) |
| Tertile 1 (n = 810) | 38.6 | (32.2, 46.2) | 1 | (ref) | 1 | (ref) |
| Tertile 2 (n = 810) | 29.9 | (24.4, 36.5) | 0.78 | (0.59, 1.02) | 0.82 | (0.61, 1.08) |
| Tertile 3 (n = 810) | 31 | (25.5, 37.7) | 0.8 | (0.62, 1.05) | 0.76 | (0.56, 1.04) |
| | All-Cause Graft Failure (GF) | | | | | |
| Uromodulin | | | | | | |
| Log 2 (n = 2430) | 65.7 | (60.8, 71.1) | 1.06 | (1, 1.12) | 1.07 | (1.01, 1.13) |
| Tertile 1 (n = 810) | 60.1 | (52.1, 69.4) | 1 | (ref) | 1 | (ref) |
| Tertile 2 (n = 810) | 66.9 | (58.5, 76.6) | 1.11 | (0.91, 1.36) | 1.12 | (0.91, 1.37) |
| Tertile 3 (n = 810) | 70 | (61.4, 79.8) | 1.13 | (0.93, 1.37) | 1.16 | (0.95, 1.43) |
| Osteopontin | | | | | | |
| Log2 (n = 2430) | 65.7 | (60.8, 71.1) | 0.96 | (0.93, 1) | 0.95 | (0.91, 1) |
| Tertile 1 (n = 810) | 71.8 | (62.9, 82) | 1 | (ref) | 1 | (ref) |
| Tertile 2 (n = 810) | 64.7 | (56.5, 74.1) | 0.89 | (0.74, 1.08) | 0.86 | (0.7, 1.05) |
| Tertile 3 (n = 810) | 61 | (53, 70.1) | 0.83 | (0.68, 1) | 0.77 | (0.61, 0.96) |

^{a}Adjusted for urine creatinine, KDRI, and the following clinical covariates: cold ischemia time (22 missing), recipient age (years), race, sex, prior kidney transplant, diabetes as the cause of end-stage kidney disease, number of human leukocyte antigen mismatches, panel reactive antibody (%), body mass index (1 missing), and pre-emptive transplant
There were no significant interactions by donor AKI status in the relationship between UMOD and dcGF and GF.

TABLE 4
Association of Donor UMOD to OPN ratio with Risk of All-Cause Graft Failure
and Death-Censored Graft Failure in the Training and Test Data Set

| | | Mean Event Rate | | | | | Mean Event Rate | | | | |
|---|---|---|---|---|---|---|---|---|---|---|---|
| Ratio of | | (95% CI), per | Unadjusted HR | | Adjusted* HR | | (95% CI), per | Unadjusted HR | | Adjusted^{a} HR | |
| UMOD | Total | 1000 person year | (95% CI) | | (95% CI) | | 1000 person year | (95% CI) | | (95% CI) | |
| to OPN | N | Death Censored Graft Failure | | | | | All Cause Graft Failure (GF) | | | | |
| | | Training Data Set | | | | | | | | | |
| >3 | 387 | 44.6 (35.0, 56.9) | 1 | (ref) | 1 | (ref) | 76.8 (63.9, 92.5) | 1 | (ref) | 1 | (ref) |
| ≤3 | 828 | 26.5 (21.5, 32.7) | 0.60 | (0.43, 0.83) | 0.57 | (0.41, 0.80) | 58.4 (50.7, 67.3) | 0.76 | (0.60, 0.96) | 0.73 | (0.57, 0.93) |
| | | Test Data Set | | | | | | | | | |
| >3 | 387 | 43.1 (33.6, 55.1) | 1 | (ref) | 1 | (ref) | 85.4 (71.7, 101.8) | 1 | (ref) | 1 | (ref) |

| | | | | | | | | | | |
|---|---|---|---|---|---|---|---|---|---|---|
| ≤3 | 828 | 29.9 (24.6, 36.5) | 0.69 | (0.51, 0.96) | 0.73 | (0.52, 1.02) | 59.3 (51.5, 68.2) | 0.69 | (0.55, 0.87) | 0.700.56, 0.88) |

There were no significant interactions between the ratio of UMOD to OPN with dcGF and GF by donor AKI status.
^{a}Adjusted for Urine Creatinine, KDRI, and the following clinical covariates: cold ischemia time (22 missing), recipient age (years), race, sex, prior kidney transplant, diabetes as the cause of end-stage kidney disease, number of human leukocyte antigen mismatches, panel reactive antibody (%), body mass index (1 missing), and pre-emptive transplant

TABLE 5a
Donor Characteristics by Tertiles of Uromodulin

| | Tertiles of Uromodulin | | | | | | |
|---|---|---|---|---|---|---|---|
| | Lower | | Middle | | Upper | | |
| | (31-1248 | | (1248-3052 | | (3065-40,400 | | |
| | ng/mL) | | ng/mL) | | ng/mL) | | |
| Donor Characteristics | N = 439 | | N = 434 | | N = 425 | | P-value |
| Age (years) | 40.66 | (14.56) | 41.92 | (14.74) | 41.75 | (14.3) | 0.33 |
| Male, n(%) | 253 | (58%) | 255 | (59%) | 276 | (65%) | 0.06 |
| Black Race, n(%) | 68 | (15%) | 72 | (17%) | 65 | (15%) | 0.85 |
| Hispanic Race, n(%) | 44 | (10%) | 50 | (12%) | 77 | (18%) | <0.001 |
| BMI (kg/m²)^{a} | 28.5 | (7.05) | 27.83 | (6.75) | 28.93 | (7.84) | 0.15 |
| Hypertension, n(%) | 134 | (31%) | 141 | (32%) | 124 | (29%) | 0.57 |
| Diabetes, n(%) | 54 | (12%) | 37 | (9%) | 39 | (9%) | 0.14 |
| Cause of Death, n(%) | | | | | | | |
| Head Trauma | 145 | (34%) | 125 | (30%) | 126 | (30%) | 0.18 |
| Anoxia | 143 | (34%) | 134 | (32%) | 148 | (35%) | |
| Stroke | 134 | (32%) | 156 | (37%) | 137 | (33%) | |
| Other | 3 | (1%) | 5 | (1%) | 10 | (2%) | |
| Hepatitis C, n(%) | 18 | (4%) | 20 | (5%) | 10 | (2%) | 0.19 |
| DCD including DBD, | 78 | (18%) | 98 | (23%) | 70 | (16%) | 0.05 |
| n(%) | | | | | | | |
| DCD, n(%) | 31 | (7%) | 57 | (13%) | 118 | (28%) | <0.001 |
| KDPI based on KDRI^{b} | 48.51 | (27.06) | 48.38 | (28.47) | 47.8 | (26.5) | 0.92 |
| Time from admission | 4.09 | (5.25) | 4.58 | (3.88) | 6.53 | (9.44) | <0.001 |
| to pronounce (days)^{c} | | | | | | | |
| Admission Creatinine | 1.15 | (0.64) | 1.08 | (0.45) | 1.06 | (0.71) | <0.001 |
| Terminal Creatinine | 1.43 | (1.15) | 1.09 | (0.69) | 0.99 | (0.51) | <0.001 |
| Donor AKIN Stage | | | | | | | |
| No AKI | 291 | (66%) | 342 | (79%) | 343 | (81%) | <0.001 |
| Stage 1 | 90 | (21%) | 62 | (14%) | 59 | (14%) | |
| Stage 2 | 27 | (6%) | 20 | (5%) | 15 | (4%) | |
| Stage 3 | 31 | (7%) | 10 | (2%) | 8 | (2%) | |
| Number of discard | | | | | | | |
| 0 | 371 | (85%) | 376 | (87%) | 385 | (91%) | 0.03 |
| 1 | 68 | (15%) | 58 | (13%) | 40 | (9%) | |

Abbreviations: AKI, acute kidney injury; BMI, body mass index; ESRD, end-stage renal disease; DCD, donor after cardiac death; DBD, donor after brain death; HLA, human leukocyte antigen; KDRI, kidney donor risk index; KDPI, kidney donor profile index
^{a}3 Donors missing BMI
^{b}3 Donors missing KDPI
^{c}13 Donors missing time from admission to pronounce (days)

TABLE 5b
Donor Characteristics by Tertiles of Osteopontin

| | Tertiles of Osteopontin | | | | | | |
|---|---|---|---|---|---|---|---|
| | Lower | | Middle | | Upper | | |
| | (53-808 | | (809-2445 | | (2448-46,100 | | |
| | ng/mL) | | ng/mL) | | ng/mL) | | |
| Donor Characteristics | N = 433 | | N = 433 | | N = 432 | | P-value |
| Age (years) | 40.67 | (14.25) | 41.74 | (14.64) | 41.91 | (14.72) | 0.41 |
| Male, n(%) | 234 | (54%) | 278 | (64%) | 272 | (63%) | 0.004 |
| Black Race, n(%) | 77 | (18%) | 58 | (13%) | 70 | (16%) | 0.20 |
| Hispanic Race, n(%) | 63 | (15%) | 59 | (14%) | 49 | (11%) | 0.36 |
| BMI (kg/m²)^{a} | 28.63 | (6.79) | 28.14 | (6.85) | 28.48 | (8) | 0.24 |
| Hypertension, n(%) | 126 | (29%) | 139 | (32%) | 134 | (31%) | 0.63 |
| Diabetes, n(%) | 48 | (11%) | 47 | (11%) | 35 | (8%) | 0.27 |
| Cause of Death, n(%) | | | | | | | |
| Head Trauma | 127 | (31%) | 132 | (31%) | 137 | (32%) | 0.46 |
| Anoxia | 153 | (37%) | 134 | (32%) | 138 | (32%) | |
| Stroke | 133 | (32%) | 149 | (35%) | 145 | (34%) | |
| Other | 3 | (1%) | 9 | (2%) | 6 | (1%) | |
| Hepatitis C, n(%) | 13 | (3%) | 16 | (4%) | 19 | (4%) | 0.55 |
| DCD including DBD, n(%) | 70 | (16%) | 82 | (19%) | 94 | (22%) | 0.11 |
| DCD, n(%) | 51 | (12%) | 75 | (17%) | 80 | (19%) | 0.02 |
| KDPI based on KDRI^{b} | 46.35 | (27.81) | 48.06 | (27.35) | 50.29 | (26.78) | 0.11 |
| Time from admission to | 6.12 | (9.1) | 4.82 | (4.97) | 4.28 | (5.14) | <0.001 |
| pronounce (days)^{c} | | | | | | | |
| Admission Creatinine | 1.13 | (0.84) | 1.08 | (0.42) | 1.08 | (0.48) | 0.68 |
| Terminal Creatinine | 1.11 | (0.86) | 1.14 | (0.77) | 1.26 | (0.91) | <0.001 |
| Donor AKIN Stage | | | | | | | |
| No AKI | 341 | (79%) | 334 | (77%) | 301 | (70%) | 0.003 |
| Stage 1 | 68 | (16%) | 61 | (14%) | 82 | (19%) | |
| Stage 2 | 8 | (2%) | 22 | (5%) | 32 | (7%) | |
| Stage 3 | 16 | (4%) | 16 | (4%) | 17 | (4%) | |
| Number of discard | | | | | | | |
| 0 | 377 | (87%) | 377 | (87%) | 378 | (88%) | 0.98 |
| 1 | 56 | (13%) | 56 | (13%) | 54 | (13%) | |

Abbreviations: AKI, acute kidney injury; BMI, body mass index; ESRD, end-stage renal disease; DCD, donor after cardiac death; DBD, donor after brain death; HLA, human leukocyte antigen; KDRI, kidney donor risk index; KDPI, kidney donor profile index
^{a}3 Donors missing BMI
^{b}3 Donors missing KDPI
^{c}13 Donors missing don adm to proc

TABLE 6a
Recipient Characteristics by Tertiles of Uromodulin

| | Tertiles of Donor Uromodulin Levels | | | | | | |
|---|---|---|---|---|---|---|---|
| | Lower | | Middle | | Upper | | |
| | (31-1248 | | (1248-3052 | | (3065-40,400 | | |
| | ng/mL) | | ng/mL) | | ng/mL) | | |
| Recipient Characteristics | N = 810 | | N = 810 | | N = 810 | | P-value |
| Age (years) | 53.6 | (14.6) | 52.77 | (15.09) | 52.37 | (14.8) | 0.13 |
| Male, n(%) | 483 | (60%) | 505 | (62%) | 504 | (62%) | 0.45 |
| Black Race, n(%) | 324 | (40%) | 306 | (38%) | 326 | (40%) | 0.53 |
| Hispanic race, n(%) | 83 | (10%) | 89 | (11%) | 107 | (13%) | 0.15 |
| Body mass index (kg/m²) | 27.94 | (5.72) | 28.08 | (5.85) | 28.08 | (5.73) | 0.70 |
| Cause of ESRD, n(%) | | | | | | | |
| Unknown/other | 173 | (21%) | 163 | (20%) | 160 | (20%) | 0.55 |
| Diabetes | 254 | (31%) | 244 | (30%) | 228 | (28%) | |
| Hypertension | 212 | (26%) | 218 | (27%) | 213 | (26%) | |
| Glomerulonephritis | 116 | (14%) | 133 | (16%) | 142 | (18%) | |
| Graft Failure | 55 | (7%) | 52 | (6%) | 67 | (8%) | |
| ESRD Duration (months) | 44.24 | (40.27) | 44.85 | (36.77) | 48.47 | (36.96) | 0.01 |
| Preemptive Transplant, n(%) | 95 | (12%) | 102 | (13%) | 77 | (10%) | 0.13 |
| Calc for KI and KP tx . . . ? | 99 | (12%) | 102 | (13%) | 114 | (14%) | 0.50 |
| Recipient Transfusions, n(%) | | | | | | | |
| No | 616 | (76%) | 600 | (74%) | 628 | (78%) | 0.41 |
| Unknown | 49 | (6%) | 58 | (7%) | 41 | (5%) | |
| Yes | 145 | (18%) | 152 | (19%) | 141 | (17%) | |
| Candidate most recent PRA | 20.75 | (35.15) | 21.6 | (34.68) | 22.11 | (35.62) | 0.58 |
| Recipient Panel Reactive | | | | | | | |
| Antibody, n(%) | | | | | | | |
| 0% | 529 | (65%) | 506 | (62%) | 510 | (63%) | |
| 1-20% | 59 | (7%) | 58 | (7%) | 61 | (8%) | |
| 21-80% | 92 | (11%) | 125 | (15%) | 109 | (13%) | |
| >80% | 130 | (16%) | 121 | (15%) | 130 | (16%) | |
| Biopsy, n(%) | 357 | (44%) | 390 | (48%) | 370 | (46%) | 0.25 |
| Kidney Pump, n(%) | 224 | (28%) | 309 | (38%) | 419 | (52%) | <0.001 |
| Cold ischemia Time | 14.76 | (7.12) | 14.87 | (7.05) | 16.24 | (7.02) | <0.001 |
| HLA mismatch level | 4.08 | (1.61) | 4.31 | (1.47) | 4.26 | (1.47) | 0.02 |
| Donor AKIN stage | | | | | | | |
| No AKI | 547 | (68%) | 637 | (79%) | 661 | (82%) | <0.001 |
| Stage 1 | 166 | (20%) | 117 | (14%) | 108 | (13%) | |
| Stage 2 | 43 | (5%) | 37 | (5%) | 29 | (4%) | |
| Stage 3 | 54 | (7%) | 19 | (2%) | 12 | (1%) | |

Abbreviations: HLA, AKIN, AKI, ESRD, PRA
1 recipient missing body mass index
50 recipients missing recipient ESRD duration
22 recipients missing information on cold ischemia time
6 recipients missing info on HLA mismatch level

TABLE 6b
Recipient Characteristics by Tertiles of Osteopontin

| | Tertiles of Donor Osteopontin Levels | | | | | | |
|---|---|---|---|---|---|---|---|
| | Lower | | Middle | | Upper | | |
| | (53-808 | | (809-2445 | | (2448-46,100 | | |
| | ng/mL) | | ng/mL) | | ng/mL) | | |
| Recipient Characteristics | N = 810 | | N = 810 | | N = 810 | | P-value |
| Age (years) | 51.87 | (15.25) | 53.55 | (15.02) | 53.32 | (14.18) | 0.05 |
| Male, n(%) | 490 | (60%) | 491 | (61%) | 511 | (63%) | 0.48 |
| Black Race, n(%) | 316 | (39%) | 301 | (37%) | 339 | (42%) | 0.15 |
| Hispanic race, n(%) | 93 | (11%) | 89 | (11%) | 97 | (12%) | 0.82 |
| Body mass index (kg/m²) | 28.2 | (5.93) | 27.95 | (5.67) | 27.96 | (5.69) | 0.86 |
| Cause of ESRD, n(%) | | | | | | | |
| Unknown/other | 168 | (21%) | 160 | (20%) | 168 | (21%) | 0.53 |
| Diabetes | 230 | (28%) | 255 | (31%) | 241 | (30%) | |
| Hypertension | 203 | (25%) | 212 | (26%) | 228 | (28%) | |
| Glomerulonephritis | 142 | (18%) | 127 | (16%) | 122 | (15%) | |
| Graft Failure | 67 | (8%) | 56 | (7%) | 51 | (6%) | |
| ESRD Duration (months) | 47 | (39.5) | 43.72 | (36.82) | 46.8 | (37.77) | 0.14 |
| Preemptive Transplant, n(%) | 83 | (10%) | 104 | (13%) | 87 | (11%) | 0.22 |
| Calc for KI and KP tx . . . ? | 118 | (15%) | 95 | (12%) | 102 | (13%) | 0.22 |
| Recipient Transfusions, n(%) | | | | | | | |
| No | 613 | (76%) | 615 | (76%) | 616 | (76%) | 0.99 |
| Unknown | 52 | (6%) | 48 | (6%) | 48 | (6%) | |
| Yes | 145 | (18%) | 147 | (18%) | 146 | (18%) | |
| Candidate most recent PRA | 22.97 | (36.49) | 21.51 | (34.97) | 19.98 | (33.89) | 0.79 |
| Recipient Panel Reactive | | | | | | | |
| Antibody, n(%) | | | | | | | |
| 0% | 520 | (64%) | 510 | (63%) | 515 | (64%) | 0.07 |
| 1-20% | 42 | (5%) | 63 | (8%) | 73 | (9%) | |
| 21-80% | 107 | (13%) | 115 | (14%) | 104 | (13%) | |
| >80% | 141 | (17%) | 122 | (15%) | 118 | (15%) | |
| Biopsy, n(%) | 348 | (43%) | 377 | (47%) | 392 | (48%) | 0.08 |
| Kidney Pump, n(%) | 292 | (36%) | 325 | (40%) | 335 | (41%) | 0.07 |
| Cold ischemia Time | 14.24 | (6.68) | 15.53 | (7.2) | 16.1 | (7.27) | <0.001 |
| HLA mismatch level | 4.12 | (1.57) | 4.19 | (1.54) | 4.33 | (1.44) | 0.03 |
| Donor AKIN stage | | | | | | | |
| No AKI | 649 | (80%) | 630 | (78%) | 566 | (70%) | <0.001 |
| Stage 1 | 123 | (15%) | 114 | (14%) | 154 | (19%) | |
| Stage 2 | 12 | (1%) | 39 | (5%) | 58 | (7%) | |
| Stage 3 | 26 | (3%) | 27 | (3%) | 32 | (4%) | |

Abbreviations: HLA, AKIN, AKI, ESRD, PRA
1 recipient missing body mass index
50 recipients missing recipient ESRD duration
22 recipients missing information on cold ischemia time
6 recipients missing info on HLA mismatch level

TABLE 7
Donor Levels of UMOD, OPN, and Urine Creatinine by AKI Status

| | | | | | AKI | | | | | | |
|---|---|---|---|---|---|---|---|---|---|---|---|
| Mean (IQR) Urine | All Donors | | No AKI | | Stage 1 | | Stage 2 | | Stage 3 | | P- |
| Markers | (N = 1298) | | (N = 976) | | (N = 211) | | (N = 62) | | (N = 49) | | value |
| UMOD (ng/mL) | 1968 | (984, 3819) | 2155 | (1093, 3983) | 1528 | (758, 3481) | 1350 | (720, 3050) | 921 | (280, 2669) | <0.001 |
| OPN (ng/mL) | 1438 | (534, 3301) | 1295 | (489, 2981) | 1665 | (567, 4630) | 2729 | (1419, 5999) | 2011 | (593, 3584) | <0.001 |
| Creatinine (mg/dL) | 36 | (14, 67) | 35 | (13, 68) | 39 | (15, 69) | 39 | (23, 67) | 33 | (21, 55) | 0.23 |
| Creatinine Corrected | 5490 | (2655, 13581) | 6082 | (2935, 15760) | 4323 | (2336, 9259) | 3046 | (1506, 8732) | 2521 | (983, 5882) | <0.001 |
| UMOD (10⁶) | | | | | | | | | | | |
| Creatinine Corrected | 4056 | (2020, 8625) | 3792 | (1953, 8071) | 4679 | (2370, 9085) | 7249 | (3203, 13422) | 5759 | (1891, 10774) | 0.004 |
| OPN (10⁶) | | | | | | | | | | | |

TABLE 8a
Donor Characteristics in the Training and Test Dataset

| | Test Dataset | | Training Dataset | | |
|---|---|---|---|---|---|
| Donor Characteristics | (N = 1215) | | (N = 1215) | | P-value |
| Age | 40.67 | (14.56) | 40.96 | (14.45) | 0.683 |
| Hispanic | 154 | (13%) | 174 | (14%) | 0.235 |
| Black Race | 193 | (16%) | 193 | (16%) | 1 |
| Male | 743 | (61%) | 738 | (61%) | 0.835 |
| Body Mass Index, kg/m² | 28.3 | (7.25) | 28.3 | (7.04) | 0.814 |
| Hypertension | 342 | (28%) | 368 | (30%) | 0.246 |
| Diabetes | 114 | (9%) | 113 | (9%) | 0.944 |
| Donor Donation After Cardiac Death | 176 | (14%) | 212 | (17%) | 0.046 |
| Expanded Criteria Donor | 209 | (17%) | 218 | (18%) | 0.631 |
| Hepatitis C virus | 43 | (4%) | 31 | (3%) | 0.157 |
| Cause of Death | | | | | |
| Head Trauma | 395 | (33%) | 370 | (31%) | 0.731 |
| Anoxia | 392 | (33%) | 397 | (34%) | |
| Stroke | 383 | (32%) | 399 | (34%) | |
| Other | 16 | (1%) | 18 | (2%) | |
| Time from admission to pronounce (days) | 5.01 | (7.16) | 5.17 | (6.52) | 0.069 |
| Admission Creatinine(mg/dL) | 1.11 | (0.57) | 1.09 | (0.61) | 0.118 |
| Terminal Serum Creatinine (mg/dL) | 1.14 | (0.79) | 1.17 | (0.87) | 0.758 |
| KDRI | 1.26 | (0.4) | 1.28 | (0.41) | 0.32 |
| KDPI | 46.3 | (27.4) | 47.39 | (26.85) | 0.329 |
| Number of Kidneys Transplanted | | | | | |
| 1 | 70 | (6%) | 96 | (8%) | 0.037 |
| 2 | 1145 | (94%) | 1119 | (92%) | |
| AKIN Stage | | | | | |
| No AKI | 937 | (77%) | 908 | (75%) | 0.039 |
| Stage 1 | 199 | (16%) | 192 | (16%) | |
| Stage 2 | 48 | (4%) | 61 | (5%) | |
| Stage 3 | 31 | (3%) | 54 | (4%) | |

^{a}1 Donor missing value for time from admission to pronounce (days)
^{b}5 Donors missing values for KDPI and KDRI

TABLE 8b
Recipient Characteristics in the Training and Test Dataset

| | Test Dataset | | Training Dataset | | |
|---|---|---|---|---|---|
| Recipient Characteristics | (N = 1215) | | (N = 1215) | | P-value |
| Age | 52.42 | (15.07) | 53.4 | (14.59) | 0.121 |
| Hispanic | 136 | (11%) | 143 | (12%) | 0.656 |
| Black Race | 495 | (41%) | 461 | (38%) | 0.158 |
| Male | 747 | (61%) | 745 | (61%) | 0.934 |
| Body Mass Index, kg/m² | 27.96 | (5.74) | 28.11 | (5.79) | 0.523 |
| Previous transplant | 157 | (13%) | 158 | (13%) | 0.952 |
| ESRD Duration in Months | 45.39 | (37.17) | 46.31 | (38.95) | 0.472 |
| ESRD Cause | | | | | |
| Other or unknown | 272 | (22%) | 224 | (18%) | 0.171 |
| Diabetes | 350 | (29%) | 376 | (31%) | |
| Hypertension | 310 | (26%) | 333 | (27%) | |
| Glomerulonephritis | 196 | (16%) | 195 | (16%) | |
| Graft failure | 87 | (7%) | 87 | (7%) | |
| Preemptive Transplant | 143 | (12%) | 131 | (11%) | 0.442 |
| Pre-Transplant Transfusions | 225 | (19%) | 213 | (18%) | 0.531 |
| Human Leukocyte Antigen Mismatch Level | 4.21 | (1.57) | 4.22 | (1.48) | 0.413 |
| Candidate Most Recent Calculated PRA | 19.89 | (34.1) | 23.08 | (36.1) | 0.105 |
| Panel Reactive Antibody (%) | | | | | |
| 0% | 786 | (65%) | 759 | (62%) | 0.158 |
| 1-20% | 98 | (8%) | 80 | (7%) | |
| 21-80% | 154 | (13%) | 172 | (14%) | |
| >80% | 177 | (15%) | 204 | (17%) | |
| TRR DIALYSIS | 1069 | (88%) | 1081 | (89%) | 0.744 |
| Primary Insurance Type | | | | | |
| Medicaid & Medicare | 896 | (74%) | 918 | (76%) | 0.305 |
| Private Insurance | 319 | (26%) | 297 | (24%) | |
| Kidney pumped | 454 | (37%) | 498 | (41%) | 0.067 |
| Pre-transplanted Dialysis (Yes/No) | 1072 | (88%) | 1084 | (89%) | 0.442 |
| Cold ischemia time (hours) | 15.27 | (6.92) | 15.31 | (7.27) | 0.834 |
| Kidney Biopsy Taken | 546 | (45%) | 571 | (47%) | 0.309 |
| Kidney Graft Failed | 161 | (13%) | 152 | (13%) | 0.586 |
| Kidney Survival Time (Days) | 1423.89 | (590.51) | 1425.74 | (593.88) | 0.883 |

1 recipient missing body mass index
6 recipients missing Human Leukocyte Antigen Mismatch Level
22 Recipients missing information on cold ischemia time

TABLE 9
Summary Table of Biopsy Deceased Donors

| | Age | Sex | Creatinine | Histologic Findings/Dx |
|---|---|---|---|---|
| ATI/ATN | 17 | M | 7.2 | — |
| (n = 6) | 57 | F | 3.1 | — |
| | 24 | F | 6.4 | PE |
| | 27 | M | 4.2 | OD |
| | 41 | F | 3.33 | MI |
| | 30 | F | 2.72 | — |
| NO ATI | 43 | F | 0.9 | recurrent nephrotic syndrome |
| (n = 4) | 48 | M | 0.8 | unremarkable allograft |
| | 51 | M | 1.2 | Glomerulomegaly/FSGS |
| | 55 | F | 1.1 | Lupus Nephritis (Class III) |

### Example 2: Initial Development of a Lateral Flow Assay

In certain embodiments, the present invention is directed to compositions and methods to ensure organ donation is successful upon implantation. As described herein, in particular embodiments, measurement and correlation of certain biomarkers, Uromodulin (UMOD) and osteopontin (OPN), has been performed in a large cohort study. The ratio of UMOD to OPN may help characterize deceased-donor kidneys and avoid AKI (acute kidney injury) after implantation. It also may help in identifying a greater population of healthy donor organs to reduce wait times for those needing transplants. In that analysis, an electro-chemiluminescent immunoassay platform by Meso Scale Discovery (MSD) in this analysis. This platform is appropriate for research and laboratory use, but is not considered field deployable. Thus, this Example is directed to the initial development efforts around a lateral flow immunoassay. In addition to UMOD and OPN, the initial development efforts also tested three other markers, TNF-alpha, IL-9, and YKL-40.

More specifically, as described below, a proof of concept study was undertaken in which pairs of commercial antibodies directed towards five biomarkers of interest (UMOD, OPN, TNF-alpha, IL-9, and YKL-40) were evaluated and configured for inclusion in lateral flow immunoassay format.

Commercial antibody affinity comparison to MSD reagents for IL-9 & TNF-Alpha. Antibody affinity (Kon/Kdis) was determined for reagents listed in Table 10 below.

| Target | Antibody Source | Antigen |
|---|---|---|
| TNF-Alpha | R&D Systems MAB610 | R&D Systems 210-TA-005 |
| | R&D Systems BAF210 | |
| | MSD capture | |
| IL-9 | R&D Systems AF209 | R&D Systems 209-ILB-010 |
| | R&D Systems AB209 | |
| | MSD capture | |
| | MSD detection | |

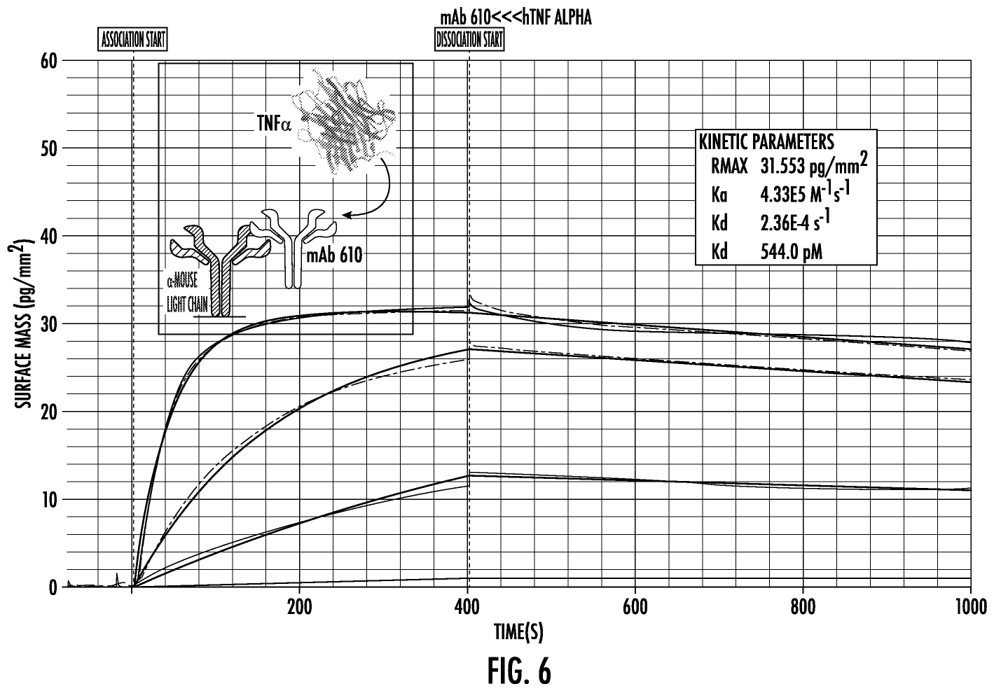

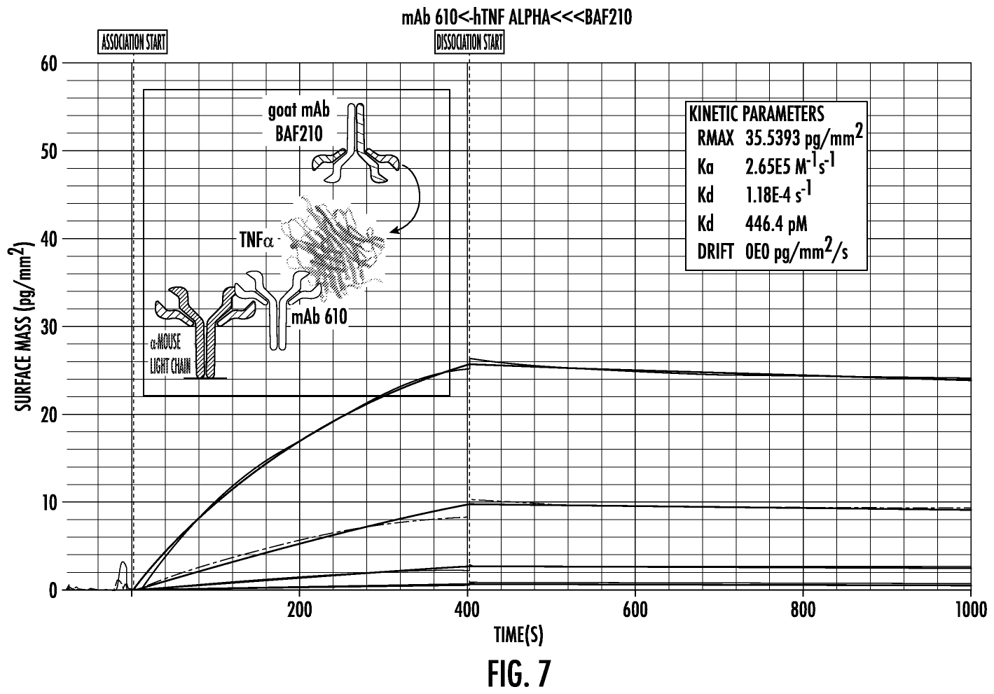

Affinity measurements were taken for the TNF-α and IL-9 antibodies from R&D Systems. The capture interaction and the pairing interaction were measured, using a species capture chip to immobilize the mouse capture mAb. The affinities for TNF-α (see FIG. 6 and FIG. 7.) were moderate and would not be expected to give <10 pg/ml test sensitivity in lateral flow format. The IL9 reagents showed no interaction at all, which was also confirmed in ELISA.

The MSD reagents were not available in sufficient quantity to analyze in this format, but were evaluated in standard ELISA. No signal was observed, which was not unexpected when compared to the highly optimized MSD platform, which uses biotin capture and electrochemiluminescence detection systems to improve sensitivity.

Antibody conjugation to gold nanoparticles. Two antibodies for each of the five target biomarkers were conjugated to colloidal gold for use in lateral flow development. Gold conjugation is the passive adsorption of antibodies onto the surface of a gold nanoparticle (generally 40 nm). To find the optimum conditions for gold conjugation, a range of buffers at differing pH levels is compared with a range of antibody loading concentrations.

The stability of the resulting gold conjugates was assessed by salt challenge, which will cause any unstable gold conjugates to crash out of solution. This can be seen visually as a color change from bright red to dark purple, but can be measured more precisely by spectrophotometry by a shift in absorption wavelength. The solution is measured at 550 nm (for stable gold) and 600 nm (aggregated gold), and the ratio reported. An Abs 550/600 ratio greater than 3.5 indicates a stable gold conjugate. Results shown in Table 11.

TABLE 11
Conjugation Concentration Optimization: Abs 550/Abs 600 Aggregation Ratios

| | Target: | | | | | | | | | |
|---|---|---|---|---|---|---|---|---|---|---|
| | UMOD | | OPN | | TNF-α | | IL-9 | | CHI3-L1 | |
| | Antibody: | | | | | | | | | |
| | AF5144 | MAB5144 | AF1433 | MAB1433 | BAF210 | MAB610 | AF209 | MAB209 | AF2599 | MAB25991 |
| Ab loading | 1 | 2 | 3 | 4 | 5 | 6 | 7 | 8 | 9 | 10 |
| 0 μg/mL | 1.2 | 1.2 | 1.2 | 1.3 | 1.3 | 1.3 | 1.3 | 1.3 | 1.3 | 1.3 |
| 5 μg/mL | 0.9 | 3.6 | 1.0 | 0.9 | 1.0 | 2.2 | 1.0 | 1.2 | 1.0 | 1.5 |
| 10 μg/mL | 2.8 | 3.7 | 2.7 | 1.0 | 1.1 | 2.4 | 3.7 | 4.0 | 1.8 | 2.8 |
| 15 μg/mL | 4.2 | 3.4 | 4.0 | 1.4 | 2.8 | 1.9 | 4.0 | 4.3 | 4.3 | 3.4 |
| 20 μg/mL | 4.4 | 4.0 | 4.1 | 1.4 | 4.0 | 1.7 | 4.0 | 4.3 | 4.2 | 3.8 |
| 25 μg/mL | 4.0 | 3.7 | 4.1 | 1.6 | 4.1 | 1.7 | 4.3 | 4.3 | 4.3 | 3.7 |
| 30 μg/mL | 4.4 | 3.9 | 4.3 | 1.4 | 4.2 | 1.5 | 4.3 | 4.0 | 4.3 | 3.9 |

Of the 10 antibodies initially investigated, 8 were successfully conjugated to gold nanoparticles, with MAB1433 and MAB610 crashing out of solution at all concentrations tested. For the remaining antibodies, a loading concentration of 15 μg/mL was deemed sufficient to form a stable gold conjugate for all but BAF210, which required 20 μg/ml.

Additional antibodies MAB14332R and MAB2091 were obtained for OPN and IL-9 respectively to replace non-functional antibodies MAB1433 and MAB209. The IL-9 antibody was found to give poor sensitivity in ELISA, and therefore was not taken forward for gold conjugation optimization. MAB14332R showed significantly better performance as the capture rather than the detection reagent, and was not able to give an aggregation ratio greater than 3.5, so a gold conjugate of this antibody was not prepared.

Selected antibody depositing on nitrocellulose and assembly of biomarker specific individual lateral flow immunoassay dipsticks. Of the ten antibodies available, 8 were plotted down to standard CN140 nitrocellulose membrane at the standard rate of 0.1 μl/mm. Antibodies were plotted at 1 mg/mL in PBS+1% sucrose for stability once dried down. The two antibodies which were not plotted down (AF1433 and BAF210) were omitted as they would have been paired with the two antibodies that did not form stable gold conjugates. The additional antibody MAB14332R was also plotted as described above.

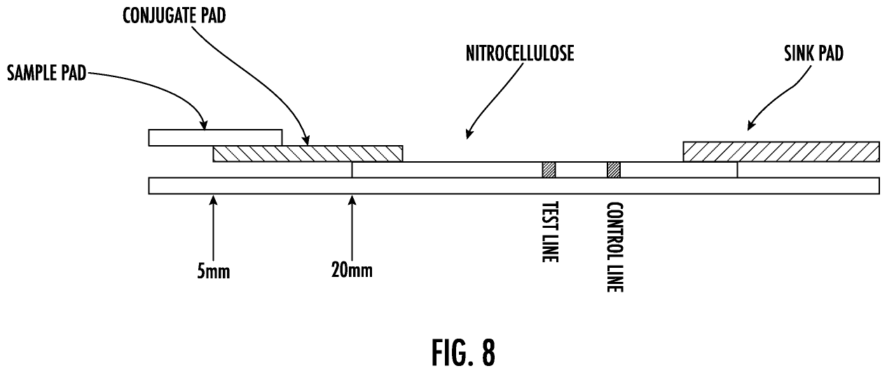

Test strips were prepared by mounting the nitrocellulose membrane onto a backing card, with a conjugate pad containing the pairing antibody sprayed down as a gold conjugate. A sample pad allows for sample filtration, and a sink pad draws the sample fluid along the test. See FIG. 8.

Testing of lateral flow dipsticks. Initial testing to find the LOD (limit of detection) was performed in a standard running buffer (PBS+0.1% Tween 20). An optical hand-held instrument (Optricon ‘Cube’ reader) was be used to assess performance of each test. In general, cube reader value greater than 10 units indicates a visible test line, while 6-10 cube units can still be faintly visible.

Test strips were initially testing with running buffer only to check for nonspecific binding (NSB), then the sample analyte was tested at the target upper limit of detection to confirm that the antibody pairing is successfully detecting the analyte2. Results are shown in Table 12.

TABLE 12
Assessment of Lateral Flow Devices

| | | Target | Target | | | Buffer Only | | Sample | | |
|---|---|---|---|---|---|---|---|---|---|---|
| | | ULOD | LLOD | | | | PBST | ULOD | | ULOD |
| Devices | Antigen | pg/ml | | NC | Gold | PBST | 1% BSA | Rep 1 | Rep 2 | ×1000 |
| 1 | UMOD | 59000000 | 5500 | AF5144 | MAB5144 | 273 | 280 | | | |
| 2 | | | | MAB5144 | AF5144 | 97 | 132 | | | |
| 3 | OPN | 47000000 | 29000 | MAB1433 | AF1433 | 125 | 161 | | | |
| 4 | TNF-α | 369 | 0.0973 | MAB610 | BAF210 | 28 | | 35 | 41 | 266 |
| 5 | IL-9 | 835 | 0.027 | AF209 | MAB209 | 15 | | 5 | 7.6 | 7 |
| 6 | | | | MAB209 | AF209 | 31 | | 37 | 30 | 37 |
| 7 | CHI3-L1 | 500000 | 0.31 | AF2599 | MAB25991 | 51 | | 249 | | |
| 8 | | | | MAB25991 | SF2599 | 16 | | 283 | 274 | |

The antibody pairings for UMOD and OPN both showed very high signal levels in buffer only. BSA was added to the running buffer as a blocking agent, but did not reduce NSB. Those devices were not used for further testing.

The TNF-α device showed some NSB, with only a small increase in signal on adding the TNF-α analyte at the target ULOD of 0.369 ng/mL. To confirm that the assay was functional, but with low sensitivity, a sample at 0.369 μg/mL was also tested: this gave a high signal.

The IL-9 devices showed no significant difference in signal between the buffer only standard and the sample analyte, even when the concentration was increased to 1000× the target ULOD of 0.835 ng/mL. This suggests that either the antibody pairing is not functional in either orientation, or that the sensitivity of the assay is very low. There was insufficient sample analyte available to test higher concentrations.

The CHI3-L1 devices gave some NSB, but did show a strong positive signal when testing the sample analyte at the target ULOD of 0.5 μg/mL. Of the two CHI3-L1 device formats, the MAB25991 capture/AF2599 detection format gave the lowest levels on NSB, so this was taken forward for further testing.

With these results in lateral flow, the decision was made to assess the functionality of the antibody pairings in ELISA, which is generally more sensitive than lateral flow.

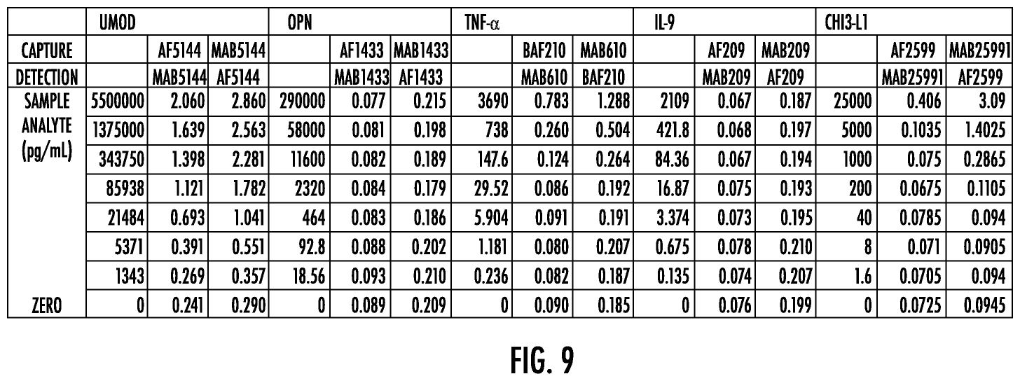

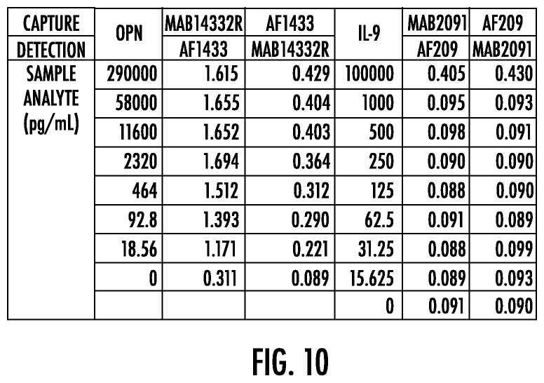

The ELISA showed that the UMOD antibodies could produce a functional assay if NSB could be reduced, while the OPN and IL9 antibodies did not pair to form a functional assay in either orientation. Both TNF-α and CHI3-L1 antibodies showed some functionality, but with an LLOD far higher than that required. See FIG. 9. The replacement OPN and IL-9 antibodies were also assessed in ELISA. See FIG. 10.

The OPN antibody pairing showed a good response with MAB14332R as the capture antibody and AF1433 as detection. The IL-9 antibody pairing was not able to detect the presence of IL-9 until the concentration was increased to 100 μg/ml; much higher than the target LLOD of 0.0973 pg/mL. It was therefore decided that work should not continue with the IL-9 antibodies.

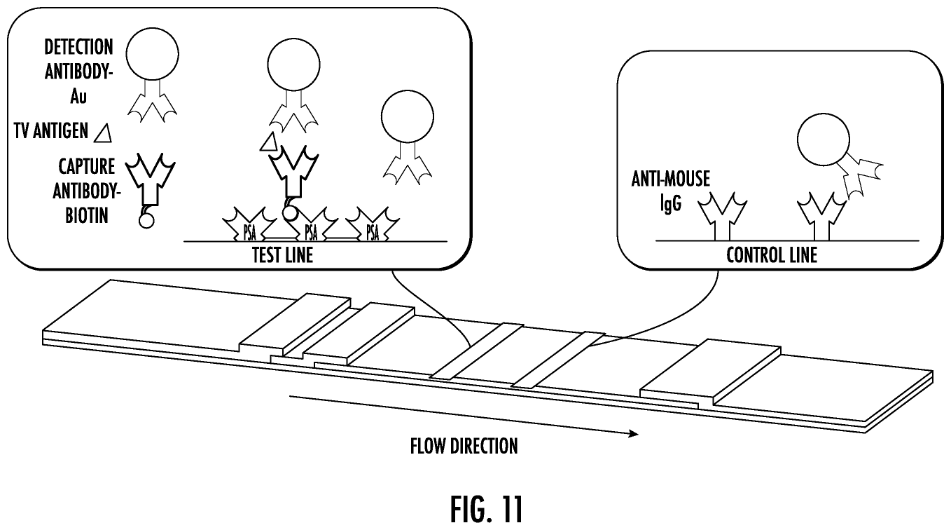

The UMOD and CHI3-L1 antibodies were taken forward for further development, using an alternative lateral flow test format in which the capture antibody is biotinylated, and travels along the test strip with the sample until it is captured by a polystreptavidin test line. When an antibody is plotted onto NC membrane only a small portion is orientated correctly to provide a functional capture reagent; by allowing the antibody to freely interact with the sample in suspension instead a significant increase in signal can be achieved while also reducing the amount of detection antibody required. See FIG. 11.

This format was able to produce a reasonably sensitive UMOD assay with minimal NSB, and a CHI3-L1 assay that shows no NSB, but is still not able to reach the LLOD (1 ng/mL instead of 0.31 pg/mL).

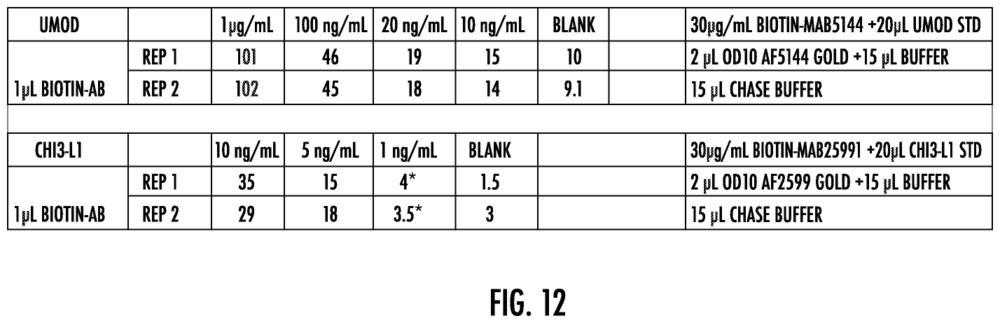

The biotin/PSA capture format was prepared using biotinylated MAB5144 (UMOD) or MAB25991 (CHI3-L1) as the capture antibody, paired with AF5144 (UMOD) or AF2599 (CHI3-L1) gold conjugate. Wet reagents were used. Biotinylated capture antibody was tested at 25, 50 and 100 ng/test to find the optimum conditions. See FIG. 12.

For the UMOD assay, the Biotin/PSA format showed an improvement on the traditional lateral flow format, with the majority of the NSB seen in the UMOD assay removed, and an LLOD of 10 ng/ml. The required LLOD is 5.5 ng/mL, however there is scope to increase sensitivity through further optimization, although it should also be noted that this testing was performed using wet reagents, and some loss of functionality is expected on drying down.

For the CHI3-L1 assay the Biotin/PSA format did not show any improvement in sensitivity, although it does offer a complete removal of NSB.

The OPN lateral flow assay showed good signal levels in PBST buffer standards, but a strong high dose hook, with samples greater than 250 ng/ml giving progressively weaker test lines. This hook occurs when there is enough of the analyte present to saturate both the capture and detection reagents separately, rather that forming a sandwich between them, and may be resolved by sample dilution. Results are shown in Table 13.

TABLE 13
Assessment of Lateral Flow Devices

| OPN Std (ng/mL) | Rep 1 | Rep 2 | Average | % CV |
|---|---|---|---|---|
| 10000 | 66 | 69 | 67.5 | 3.14 |
| 1000 | 171 | 163 | 167 | 3.39 |
| 500 | 180 | 197 | 188.5 | 6.38 |
| 250 | 196 | 188 | 192 | 2.95 |
| 50 | 165 | 155 | 160 | 4.42 |
| 25 | 137 | 143 | 140 | 3.03 |
| 5 | 70 | 76 | 73 | 5.81 |
| PBST | 15 | 14 | 14.5 | 4.88 |

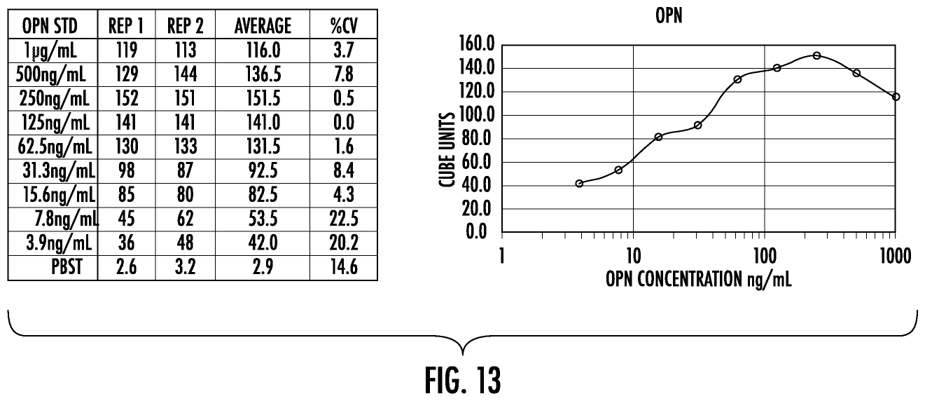

When testing the running buffer only, a visible level of background signal was observed due to nonspecific binding (NSB) gold conjugate to the test line. Standard blocking agents such as BSA and human anti-mouse antibodies (HAMA), or increasing the Tween concentration of the running buffer were not able to significantly reduce the NSB, but it was found that by halving the capture antibody concentration from lmg/mL to 0.5 mg/mL plotted at 0.05 μL/mm the NSB was removed completely while still retaining a high level of sensitivity. See FIG. 13.

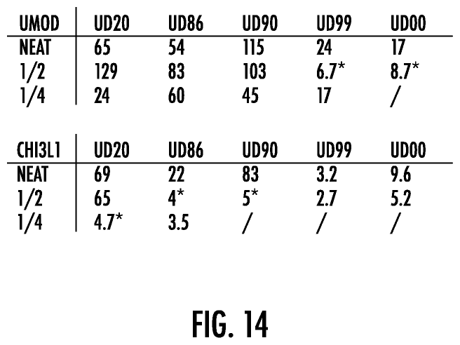

Urine testing: UMOD & CHI3-L1 Devices. UMOD and CHI3-L1 lateral flow devices were assembled using dried down materials and used to test five in-house urine samples, neat and at ½ and ¼ dilution in PBST3. Results shown in FIG. 14.

The UMOD assay gave relatively high signals for most of the samples tested, suggesting that there is a high level of OMUD present in healthy samples. The three higher samples all showed an increase, or only a slight decrease in signal on the initial ½ dilution, followed by a greater reduction in signal following further dilution. This ‘hook effect’ may be caused by matrix interference, or by an excess of the antigen which can prevent the formation of a sandwich by saturating both the gold detection reagent and the capture line.

The CHI3-L1 assay gave lower signals for most of the samples tested, which tended to dilute out rapidly.

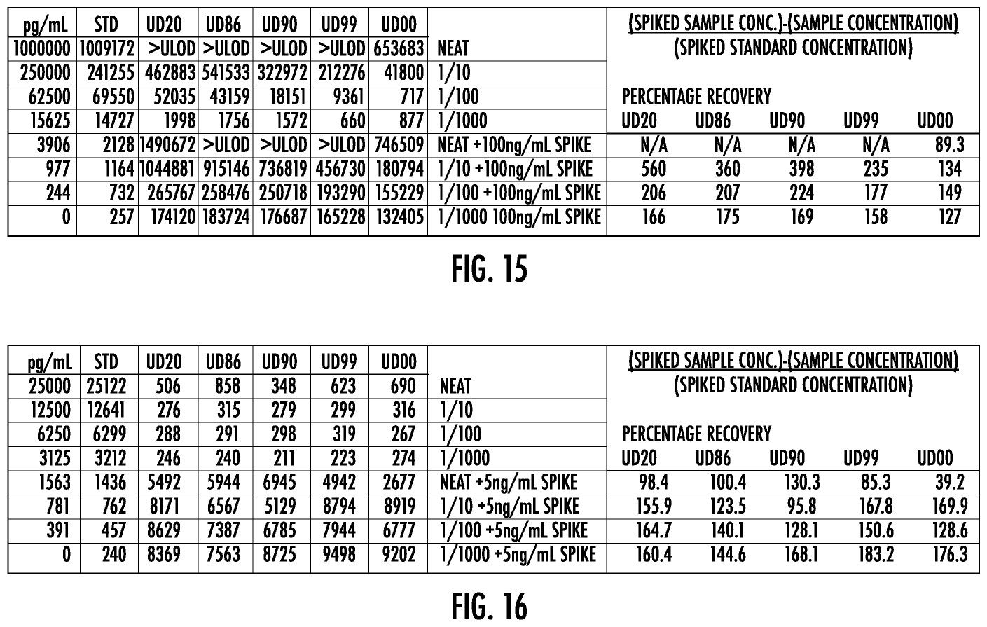

To better characterize these samples, an ELISA was performed for both assays, with the samples tested neat and at 1/10, 1/100 and 1/1000 dilution. Each sample was also spiked with a mid-range concentration of the antigen to determine matrix effects on sample recovery4. See results in FIGS. 15 and 16.

The UMOD ELISA results appear to show a similar pattern to the lateral flow test, with the two lowest samples in ELISA (UD99 and UD00) also giving the lowest signal levels. However, the concentrations of UMOD measured in those samples suggests that higher signals would have been expected in LF, indicating that there may be some matrix interference. However, the high level of variability in spike recovery suggests that the comparison between the buffer standard and the urine samples may not be reliable.

The CHI3-L1 ELISA showed reasonably good spike recovery, suggesting that quantitation of the urine samples against a buffer standard curve can be considered accurate. The results showed low levels of CHI3-L1 in healthy urine (˜0.3-0.7 ng/mL), which would not be expected to be detected by the LF, which had a LLOD of 1 ng/mL. The presence of some higher signals, e.g. for sample UD20 and UD90 at 69 and 83 cube units, therefore suggests that matrix issues may be causing nonspecific binding.

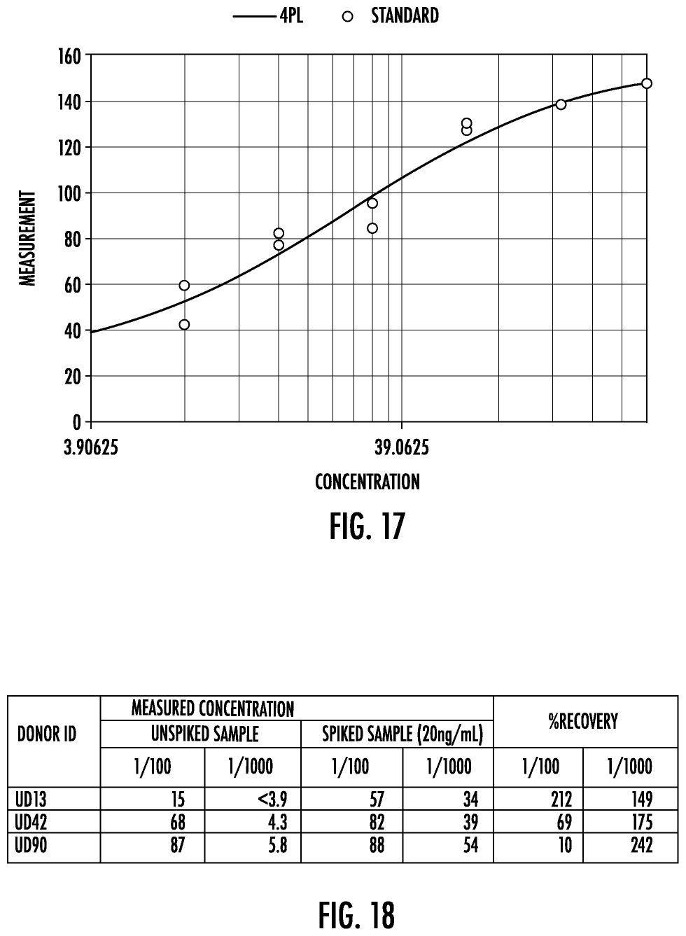

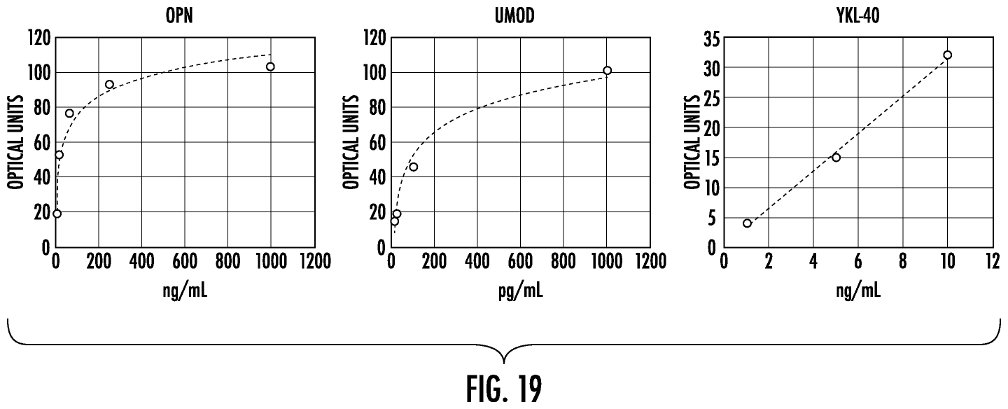

Urine testing: OPN Devices. OPN lateral flow devices were used to test three urine samples at 1/100 and 1/1000 dilution. Samples were run both unspiked and spiked with 20 ng/mL OPN. A standard curve in buffer was run in duplicate (FIG. 17), and the spiked and unspiked samples were interpolated against it to find the % recovery (FIG. 18).

For two of the urine samples, the 1/100 dilution still gave relatively strong signals and the OPN spike was difficult to detect. For the 1/1000 dilution, unspiked samples were either at the lower end or below the lower limit of the standard curve, suggesting that the optimum urine dilution lies between the two dilutions tested. Nevertheless, the OPN spike was detected, with % recovery ranging from 149 to 242%, which is reasonable given that the assay is unoptimized, and the standard curve was only run in duplicate.

Conclusions. A proof of concept study was performed in which pairs of commercial antibodies directed towards five biomarkers of interest (UMOD, OPN, TNF-alpha, IL-9, and YKL-40) were evaluated and configured for inclusion in lateral flow immunoassay format.

The antibodies available for the IL-9 and TNF-α did not display the performance required to develop a successful lateral flow test.

The UMOD and CHI3-L1 antibodies were taken forward for further development, using an alternative lateral flow test format in which the capture antibody is biotinylated, and travels along the test strip with the sample until it is captured by a polystreptavidin test line. This format was able to produce a reasonably sensitive UMOD assay with minimal NSB, and a CHI3-L1 assay that shows no NSB, but is still not able to reach the target LLOD (1 ng/mL instead of 0.31 pg/mL).

The OPN antibodies initially investigated showed poor performance, but with an alternative antibody a standard lateral flow test was developed which displayed the required LLOD.

Testing in urine showed some matrix interference all three assays, with some dilution generally required to both reduce the interference and bring the samples to within the analytical range of the relevant assay.

Forty prototype devices per analyte have been prepared for further evaluation.

1. A method comprising the step of measuring the amount of uromodulin (UMOD) and osteopontin (OPN) in a urine sample obtained from a deceased person.

2. The method of claim 1, further comprising the step of measuring the amount of YKL-40.

3. The method of claim 1, wherein the measuring step is performed using a lateral flow device.

4. A method for assessing viability of a kidney for transplantation comprising the step of measuring the amount of UMOD and OPN in a urine sample obtained from a deceased donor using a point-of-care lateral flow device, wherein ratio of UMOD:OPN of ≤3 indicates the kidney is viable for transplantation and a ratio of UMOD:OPN of >3 indicates the kidney is not viable for transplantation.

5. The method of claim 4, wherein the lateral flow device is a dipstick assay.

6. A method for assessing viability of a kidney of a deceased donor for transplantation comprising the steps of:
(a) contacting a urine sample obtained from a deceased donor with a first antibody and a second antibody on a lateral flow device, wherein the first antibody specifically binds UMOD and the second antibody specifically binds OPN; and
(b) detecting the amount of UMOD and OPN,
wherein a ratio of UMOD:OPN of ≤3 indicates the kidney is viable for transplantation and ratio of UMOD:OPN of >3 indicates the kidney is not viable for transplantation.

7. The method of claim 6, wherein the lateral flow device is a dipstick assay.

8. A kit for assessing kidney viability for transplantation comprising:
(a) a first capture agent that specifically binds UMOD present in a sample obtained from the kidney donor;
(b) a second capture agent that specifically binds OPN present in a sample obtained from the kidney donor; and
(c) instructions for performing a method of assessing kidney viability for transplantation.

9. The kit of claim 8, wherein the kidney donor is deceased.

10. The kit of claim 8, wherein the sample is a urine sample.

11. The kit of claim 8, wherein the first capture agent and the second capture agent are or are capable of being detectably labeled.

12. The kit of claim 8, further comprising:
(d) a first detection agent that detects the first capture agent bound to UMOD; and
(e) a second detection agent that detect the second capture agent bound to OPN.

13. The kit of claim 8, further comprising a third capture agent that specifically binds YKL-40 present in a sample obtained from the kidney donor.

14. The kit of claim 13, wherein the third capture agent is or is capable of being detectably labeled.

15. The kit of claim 13, further comprising a third detection agent that detects the third capture agent bound to YKL-40.

16. The kit of claim 8, further comprising a solid support on which to perform the assay.

17. A lateral flow multiplex assay device comprising:
(a) a sample pad configured to receive a urine sample from a deceased kidney donor;
(b) a conjugate pad comprising a first detection conjugate comprising an agent that specifically binds UMOD and a second detection conjugate comprising an agent that specifically binds OPN; and
(c) at least one detection zone comprising a test line.

18. The lateral flow multiplex assay device of claim 17, wherein the first detection conjugate and the second detection conjugate are antibodies.

19. The lateral flow multiplex assay device of claim 18, wherein the antibodies are biotinylated.

20. The lateral flow multiplex assay device of claim 19, wherein the test line comprises immobilized streptavidin particles.
